(19) United States
(12) Patent Application Publication
(10) Pub. No.: US 2010/0189695 A1
(43) Pub. Date: Jul. 29, 2010

(54) IRAK-1 AS REGULATOR OF DISEASES AND DISORDERS

(76) Inventors: Liwu Li, Blacksburg, VA (US); Urmila Maitra, Blacksburg, VA (US); Lu Gan, Blacksburg, VA (US)

Correspondence Address: NEW RIVER VALLEY INTELLECTUAL PROPERTY LAW, P.O. BOX 10944, BLACKSBURG, VA, 24062, US

(21) Appl. No.: 12/511,006

(22) Filed: Jul. 28, 2009

Related U.S. Application Data
(60) Provisional application No. 61/084,232, filed on Jul. 28, 2008.

(51) Int. Cl. A61K 35/12; A61P 29/00; A61P 9/12; A61P 9/10; A61P 3/10; A61P 13/12; A61P 7/00; G01N 33/573; C12Q 1/68

(52) U.S. Cl. 424/93.7; 435/7.4; 435/6

(57) ABSTRACT

The present invention provides methods and compositions for treatment of diseases and disorders. More specifically, the invention for the first time shows a link between IRAK-1 and phosphorylation of proteins involved in cardiovascular disease, diabetes, neurodegeneration, and associated diseases and disorders and complications. Typically, the diseases and disorders involve an inflammatory component. Assays for bioactive substances affecting IRAK-1 regulated progression of inflammation and diseases and disorders involving inflammation are also disclosed.

### CROSS-REFERENCE TO RELATED APPLICATIONS

This application relies on and claims the benefit of the filing date of U.S. provisional patent application No. 61/084,232, filed 28 Jul. 2008, the entire disclosure of which is incorporated herein by reference.

### STATEMENT OF GOVERNMENT INTEREST

This invention was made partially with U.S. Government support from the United States National Institutes of Health under Contract No. AI64414. The U.S. Government has certain rights in the invention.

### BACKGROUND OF THE INVENTION

1. Field of the Invention

The present invention relates to the fields of molecular biology, biochemistry, and health. More specifically, the invention relates to discovery of the role of Interleukin-1 Receptor Associated Kinase-1 (IRAK-1) in development of diseases and disorders, and characterization of the kinase for development of treatments for those diseases and disorders.

2. Description of Related Art

IRAK-1 was initially discovered as a kinase forming a close association with the intracellular domain of interleukin-1 receptor. Because IL-1 is one of the critical inflammatory cytokines, IRAK-1 has since drawn great attention in the inflammation field. The significance of IRAK-1 was further elevated when it was later found to be shared by the Toll-Like-Receptor (TLR) mediated innate immunity signaling pathways. Recently, studies have shown that other pathways, such as GPCR mediated pathway, CD26 signaling, and the insulin signaling pathway might all share IRAK-1 as one of their critical signaling components. Several homologues have been discovered, including IRAK-2, IRAK-M, and IRAK-4.

The physiological substrates of IRAK related molecules have not been clearly defined. Initial characterizations of IRAK related molecules pointed out their involvement in NFκB activation. However, further detailed studies employing transgenic mice have since demonstrated unique and distinct functions for each IRAK member. For example, IRAK-4 was shown to be the key player activating NFκB. In contrast, IRAK-M was demonstrated to be a suppressor of NFκB activation. On the other hand, IRAK-1 is not directly involved in activating NFκB, and therefore performs a distinct function.

The unique biochemical and cellular functions of various IRAK members correlate well with various phenotypes manifested by transgenic mice as well as humans harboring genetic variations. Due to the role of IRAK-4 in mediating pathogen-induced NFκB activation, deletion of IRAK-4 in mice causes severe deficiency in host defense toward infection. Humans with IRAK-4 genetic deficiencies are also extremely prone to various infections. IRAK-M deficient mice have elevated osteoclast function and excessive bone loss due to their failure to down-regulate NFκB, heart disease and failure, sepsis, and diabetes. The molecular mechanisms explaining IRAK-1 function have yet to be clearly defined.

The IRAK-1 molecule contains an N-terminal death domain with a central serine/threonine rich region, and a C-terminal serine/threonine rich region (see FIG. 1). Following various stimulations such as IL-1 (via IL-1R), lipopolysaccharide (LPS, via TLR4), and bacterial lipoprotein (Pam3CSK4, via TLR2), IRAK-1 undergoes phosphorylation, activation, and subsequent ubiquitination and degradation. IRAK-1 can also undergo sumoylation and translocation into the cell nucleus. Thus far no physiological substrates for IRAK-1 have been identified. However, IRAK-1 may serve as its own kinase, autophosphorylating itself. The region of IRAK-1 that can be autophosphorylated contains five serine/proline motifs (see FIG. 2). The crystal structure of IRAK-1 catalytic domain was recently defined. Based on the structural prediction, it has been speculated, but not confirmed, that IRAK-1 may prefer to phosphorylate serine residues immediately preceding a proline residue.

The inventor has recognized that there exists a need to understand the biochemical bases and mechanisms for diseases and disorders that affect humans and other animals and that involve IRAK-1. Through this understanding, treatment regimens and drugs may be developed to treat the diseases and disorders.

### SUMMARY OF THE INVENTION

The present invention addresses needs in the art by providing an understanding of the biochemical and biophysical bases of certain diseases and disorders that relate to the function of IRAK-1. More specifically, the present invention identifies IRAK-1 as an important protein that is involved in regulation of the development of a variety of diseases and disorders that affect the quality of health of humans and other animals. The present invention identifies Interleukin-1 Receptor Associated Kinase-1, also known as IRAK, IRAK1, and IRAK-1 (Genbank Accession: NP_{—}001560) as a protein kinase that is involved in regulation of biochemical pathways that are known to be associated with certain diseases and disorders. More specifically, the present invention shows that IRAK-1 is a critical protein kinase involved in regulating the activities of several important transcription factors contributing to the pathogenesis of inflammation, heart hypertrophy, hypertension, and atherosclerosis. Proper IRAK-1 function is required to prevent the pathogenesis of inflammation, hypertrophy, hypertension, and atherosclerosis, as well as other related complications, such as diabetes, lupus, kidney injury, and sepsis. Likewise, inhibition of IRAK-1 function can be advantageous in limiting the negative aspects of certain diseases and disorders associated with inflammation.

The invention also encompasses the use of genetic variations of IRAK-1 gene to serve as diagnostic markers for human cardiovascular diseases, including hypertension, atherosclerosis, and other complications such as diabetes. Genetic variations in human IRAK-1 gene are closely linked with these diseases, and include rare single nucleotide polymorphisms (see, for example, Single Nucleotide Polymorphism (SNP) Identification Numbers: rs11465829, rs10127175, rs1059703, rs11465830, rs3027903, and rs3027907). Humans carrying one or multiple variations have the highest risk of developing cardiovascular diseases and diabetes.

The present invention shows that IRAK-1 has multiple substrates in various different biochemical pathways, many of which are involved in diseases and disorders. IRAK-1 has thus been discovered as a key regulatory element for certain diseases and disorders, and can be used as a diagnostic marker and as a target for assays to identify agents that disrupt its physiological activity and interfere with its function in disease progression. For example, it is disclosed herein that IRAK-1 is involved in the regulation of the activity of transcription factors of the ATF-1/CREB family of proteins. It is also disclosed that IRAK-1 is involved in the regulation of activity of STATs, such as STAT-3, as well as C/EBP β/δ. Similar to its role in activating NFκB, IRAK-1 acts on these proteins to promote production of inflammatory mediators, NOX-1, and MCP-1. Further, the present invention discloses that IRAK-1 is involved in the regulation of activity of the NFAT (nuclear factor of activated T-cells) family of proteins, RAR, and LXR, ultimately leading to regulation of ABCA1 and ABCG1 in macrophages, the differentiation of T regulatory cells as well as Th17 cells, and fatty acid oxidation in metabolic cells.

It is also disclosed herein that IRAK-1 is involved in the regulation of the activity of PH (Pleckstrin-Homology) domain-containing proteins, such as PDK-1 (3-phosphoinositide-dependent protein kinase 1), Akt/PKB (protein kinase B), IRS (insulin receptor substrate), and small GTPase-activating proteins, as well as VASP (vasodilator-stimulated phosphoprotein) and other EVH-1 domain containing proteins. It is further disclosed herein that IRAK-1 is involved in regulation of the activity of Tau protein, and thus has a role in maintaining cell structure and in diseases involving neurodegeneration. While not being limited to any particular substrate binding or phosphorylation motif, data shows that IRAK-1 can phosphorylate substrates that contain one or more Serine-Proline rich motifs. For example, the substrates that are specifically exemplified herein (e.g., Tau, IRS-1, NFAT, small GTPase activating protein) contain such a motif.

In one aspect, the invention provides a method of affecting the phosphorylation state of a target protein involved in a disease or disorder. In general, the method comprises affecting the ability of an IRAK-1 protein or fragment thereof to contact the target protein, wherein the amount of contact of the two proteins is related to the amount of phosphorylation of the target protein. In embodiments, the IRAK-1 protein has protein kinase activity for the target protein, and contact of the IRAK-1 protein results in phosphorylation of the target protein, which affects the target protein's activity. In embodiments, the IRAK-1 protein does not have protein kinase activity for the target protein, and contact of the IRAK-1 protein with the target protein reduces phosphorylation of the target protein by other proteins. The method has applicability both in vitro and in vivo. For example, in vitro, the method can be a research method to study interaction of two or more proteins or inhibitors of protein-protein interactions. For example, it can be a method of drug discovery. Alternatively, the method can be practiced in vivo as a therapeutic or prophylactic method of treating a subject having or susceptible to developing a disease or disorder involving a phosphorylation state of an IRAK-1 substrate, for example. In embodiments, the target protein is an NFAT family member, a protein having a PH motif, or a Tau protein.

In another aspect, the invention provides a method of treating a patient having or susceptible to developing a disease or disorder involving IRAK-1 kinase activity. In general, the method comprises contacting a cell comprising a protein that has an activity that is regulated by IRAK-1 with a substance that alters the level or activity of the protein as a result of phosphorylation by IRAK-1, wherein the altered protein activity results in a detectable change in at least one clinical symptom of the disease or disorder or reduces or prevents the likelihood of development of at least one clinical symptom of the disease or disorder. The substance can cause an increase in phosphorylation due to IRAK-1 or a decrease in phosphorylation due to IRAK-1. The substance may be IRAK-1 or a fragment thereof having the desired activity. Alternatively, the substance may be a substance that affects the activity or expression of IRAK-1. In embodiments, the target protein is an NFAT family member, a protein having a PH motif, or a Tau protein.

In an additional aspect, the invention provides a method of phosphorylating one or more NFAT family member proteins. In general, the method comprises exposing one or more NFAT member proteins to an IRAK-1 protein under conditions that allow contact of the IRAK-1 protein and the NFAT protein, resulting in phosphorylation of the NFAT protein by the IRAK-1 protein. In a similar vein as in other aspects, in embodiments, the IRAK-1 protein is a polypeptide or peptide fragment of a full-length IRAK-1 protein, which has NFAT protein kinase activity (e.g., contains residues 1—about 521 of an IRAK-1 protein). In embodiments, the NFAT protein family member is NFAT-1, NFAT-2, NFAT-3, or NFAT-4. In embodiments, the method includes providing the IRAK-1 protein. In other embodiments, the method comprises causing expression of an IRAK-1 protein. In yet other embodiments, the method comprises causing pre-existing IRAK-1 to become available to contact the NFAT protein. The method may be practiced in vitro, for example in a cell-free system or in a controlled laboratory environment (e.g., tissue culture plate, microtiter plate), or in vivo, for example in an animal subject (also referred to herein as a patient, person, or animal).

In a further aspect, the invention provides a method of treating a patient having or susceptible to developing a disease or disorder involving the activity of an NFAT family member. In general, the method comprises exposing at least one cell of the patient to a substance that alters the amount of an un-phosphorylated NFAT family member protein in the cell by altering phosphorylation of NFAT proteins in the cell, wherein phosphorylation results in a change in the activity of the NFAT protein. In embodiments, the method comprises administering to the patient an amount of IRAK-1 protein or antagonist of IRAK-1 protein that is sufficient to alter the amount of un-phosphorylated NFAT protein to a level that results in a change in a detectable clinical symptom of the disease or disorder. As with other aspects, the IRAK-1 protein may be a full-length protein or a fragment thereof having protein kinase activity, and preferably NFAT protein kinase activity. Likewise, the NFAT protein may be any NFAT family member protein. Further, the method may be therapeutic or prophylactic. Furthermore, the methods also can comprise administering to the patients compound(s) or substance(s) that can alter IRAK-1 activities, such as the synthetic peptide triacylated Cys-Ser-Lys-Lys-Lys-Lys (Pam₃CSK₄), lipopolysaccharide (LPS), lipid A derivatives, and others.

In yet a further aspect, the invention provides a method of reducing or blocking phosphorylation of one or more proteins having a Pleckstrin Homology (PH) domain. In general, the method comprises exposing one or more PH domain-containing proteins to an IRAK-1 protein lacking protein kinase activity, a fragment thereof lacking protein kinase activity, or in the presence of a substance that inhibits or blocks contact of the IRAK-1 protein and the PH domain-containing protein under conditions that allow contact of the IRAK-1 protein and the PH domain-containing protein, resulting in a reduction or abolition of phosphorylation of the PH domain-containing protein by the IRAK-1 protein. In embodiments, the IRAK-1 protein is a polypeptide or peptide fragment of a full-length IRAK-1 protein, which is not capable of binding to the target PH domain-containing protein. In other embodiments, the IRAK-1 protein is a polypeptide or peptide fragment of a full-length IRAK-1 protein, which is capable of binding to the target PH domain-containing protein, but not capable of phosphorylating the target protein. In yet other embodiments, the method comprises exposing the IRAK-1 protein, the target PH domain-containing protein, or both, to a substance that interferes with contact between the IRAK-1 protein and the PH domain-containing protein. In embodiments, the PH domain-containing protein is PDK-1, PKB, IRS, or a small GTPase activating protein. In embodiments, the PH domain-containing protein is involved in regulation or development of diabetes. In embodiments, the inhibitor is a fragment of IRAK-1. The method may be practiced in vitro, for example in a cell-free system or in a controlled laboratory environment (e.g., tissue culture plate, microtiter plate), or in vivo, for example in an animal subject (also referred to herein as a patient, person, or animal).

In yet another aspect, the invention provides a method of treating a patient having or susceptible to developing a disease or disorder involving the activity of a PH domain-containing protein. In general, the method comprises exposing at least one cell of the patient to a substance that reduces the amount of a phosphorylated target PH domain-containing protein in the cell by reducing or eliminating phosphorylated forms of the target protein by reducing or blocking the interaction of IRAK-1 on the target protein. Reducing or blocking the interaction reduces the amount of phosphorylated PH domain-containing protein, and reduces or eliminates the disease or disorder, a detectable clinical symptom, or the likelihood of development. In embodiments, the method comprises administering to the patient an amount of an IRAK-1 protein or fragment thereof lacking kinase activity on the target PH domain-containing protein that is sufficient to reduce the amount of phosphorylated target protein to a level that results in a change in a detectable clinical symptom of the disease or disorder. As with other aspects, the IRAK-1 protein may be a full-length protein or a fragment thereof lacking protein kinase activity. In other embodiments, the method comprises administering to the patient an amount of a PH domain-containing protein or fragment thereof that is sufficient to bind to an IRAK-1 protein and reduce the amount of phosphorylated target PH domain-containing protein to a level that results in a change in a detectable clinical symptom of the disease or disorder. As with other aspects, the PH domain-containing protein may be a full-length protein or a fragment thereof having the ability to bind to IRAK-1. The PH domain-containing protein may be any such protein, including, but not limited to those involved in response to insulin. Further, the method may be therapeutic or prophylactic.

In yet an additional aspect, the invention provides a method of reducing or blocking phosphorylation of a Tau protein, such as one in a neuron. In general, the method comprises exposing a Tau protein to an IRAK-1 protein lacking protein kinase activity, a fragment thereof lacking protein kinase activity, or in the presence of a substance that inhibits or blocks contact of the IRAK-1 protein and the Tau protein under conditions that allow contact of the IRAK-1 protein and the Tau protein, resulting in a reduction or abolition of phosphorylation of the Tau protein by the IRAK-1 protein. In embodiments, the IRAK-1 protein is a polypeptide or peptide fragment of a full-length IRAK-1 protein, which is not capable of binding to the target Tau protein. In other embodiments, the IRAK-1 protein is a polypeptide or peptide fragment of a full-length IRAK-1 protein, which is capable of binding to the target Tau protein, but not capable of phosphorylating the target protein. In yet other embodiments, the method comprises exposing the IRAK-1 protein, the target Tau protein, or both, to a substance that interferes with contact between the IRAK-1 protein and the Tau protein. In embodiments, the Tau protein is involved in regulation or development of a neurological disease, such as but not limited to Alzheimer's Disease (AD) and Parkinson's Disease. In embodiments, the inhibitor is a fragment of IRAK-1. The method may be practiced in vitro, for example in a cell-free system or in a controlled laboratory environment (e.g., tissue culture plate, microtiter plate), or in vivo, for example in an animal subject.

In another aspect, the invention provides a method of treating a patient having or susceptible to developing a disease or disorder involving the activity of a Tau protein. In general, the method comprises exposing at least one cell of the patient to a substance that reduces the amount of a phosphorylated target Tau protein in the cell by reducing or eliminating phosphorylated forms of the target protein by reducing or blocking the interaction of IRAK-1 on the target protein. Reducing or blocking the interaction reduces the amount of phosphorylated Tau protein, and reduces or eliminates the disease or disorder, a detectable clinical symptom, or the likelihood of development. In embodiments, the method comprises administering to the patient an amount of an IRAK-1 protein or fragment thereof lacking kinase activity on the target Tau protein that is sufficient to reduce the amount of phosphorylated target protein to a level that results in a change in a detectable clinical symptom of the disease or disorder. As with other aspects, the IRAK-1 protein may be a full-length protein or a fragment thereof lacking protein kinase activity. In other embodiments, the method comprises administering to the patient an amount of a Tau protein or fragment thereof that is sufficient to bind to an IRAK-1 protein and reduce the amount of phosphorylated target Tau protein to a level that results in a change in a detectable clinical symptom of the disease or disorder. The Tau protein may be a full-length protein or a fragment thereof having the ability to bind to IRAK-1; however, to improve availability in brain tissue, the protein is preferably relatively short, such as a peptide having fewer than 100 residues. Further, the method may be therapeutic or prophylactic.

Full length IRAK-1 is expressed in most of the human tissues except the brain tissue where it is absent and a shorter form is present. This expression pattern might help to keep brain tissue in an immune-privileged state. Intriguingly, in previous work, the present inventor and his colleagues documented that aged human brains (>70 years old) start to express the full-length IRAK-1 form. This fact is closely correlated with the higher risk of Alzheimer's and other neurological diseases accompanied with the aging process. The fact that IRAK-1 contributes to Tau protein phosphorylation, and that Tau phosphorylation has been closely linked with neuronal cell malfunction can explain the correlation between the higher expression levels of full-length IRAK-1 in aged human brains and the higher risks of various neurological diseases. The invention thus encompasses the detection of full-length IRAK-1 transcript in brain tissue as a marker for neurological diseases.

In yet another aspect, the invention provides a composition. In general, the composition is useful for performing a method according to at least one aspect of the invention. For example, the composition may comprise an isolated or purified (at least to some extent) IRAK-1 protein or fragment thereof having protein kinase activity. Alternatively, it may comprise an isolated or purified IRAK-1 protein lacking kinase activity for a particular target protein. Likewise, it might be a full-length, fragment, or otherwise mutant form of an IRAK-1 substrate protein, which can be used to titrate the activity of an IRAK-1 protein in vitro or in vivo. Typically, the composition comprises a protein as described above and at least one other substance that is biologically tolerable. Typically, where the composition comprises a protein or fragment thereof, the composition comprises sufficient protein or fragment to enter a target cell when exposed to the cell. In embodiments, the composition is a pharmaceutical composition suitable for administration to subjects in need thereof, such as one comprising pharmaceutically acceptable excipients, carriers, buffers, salts, and the like. In some embodiments, it is preferred that the protein or fragment is incapable of traversing the blood-brain-barrier, whereas in other embodiments, it is preferred that the protein or fragment is capable of doing so.

In another aspect, the invention provides a method of identifying substances that affect the development or progression of a disease or disorder associated with IRAK-1. In general, the method comprises exposing IRAK-1 to a substance and determining whether the substance has an effect on binding of IRAK-1 to a substrate or the phosphorylating activity of IRAK-1. Antibodies to IRAK-1 and its substrates are available to the public, and can be used to identify binding of IRAK-1 to its substrates (e.g., by way of immuno co-precipitation). IRAK-1 is a kinase, and assays for its activity are known in the art. According to the method of the invention, the activity of IRAK-1 is assayed using a known kinase assay and a substrate discussed below. Comparison of the activity of IRAK-1 in the presence and absence of the substance allows one to determine if the substance has a specific effect on IRAK-1 activity on selected and highly targeted downstream targets as identified herein.

In an alternative method according to this aspect, the invention provides a method of identifying substances that affect the development or progression of a disease or disorder associated with IRAK-1. In general, the method comprises exposing ATF-1/CREB, STAT3, CEBPβ/δ, RAR, LXR, or a member of the NFAT family to a substance and determining whether the substance has an effect on binding of the ATF-1/CREB, STAT3, CEBPβ/δ, RAR, LXR, or a member of the NFAT family to IRAK-1, or affects the kinase activity of IRAK-1 on these substrates. According to the method of the invention, the activity of IRAK-1 can be assayed using a known kinase assay. Comparison of the activity of IRAK-1 in the presence and absence of the substance allows one to determine if the substance has an effect on IRAK-1 activity.

Alternatively, the methods comprise exposing cells to a substance and determining whether the substance can modulate the activation status of ATF-1/CREB, STAT3, CEBPβ/δ following lipopolysaccharide (LPS) treatment in an IRAK-1 dependent fashion. Likewise, the methods can comprise exposing cells to a substance and determining whether the substance can modulate the activation status of ATF-1/CREB, STAT3, CEBPβ/δ, RAR family proteins, LXR member proteins, and PPAR family member proteins following stimulation with nuclear receptor agonists such as all trans retinoic acid (ATRA) or other synthetic agonists in an IRAK-1 dependent fashion. Likewise, the methods comprises exposing cells to a substance and determining whether the substance may modulate the activation status of ATF-1/CREB, STAT3, CEBPβ/δ following sequential treatments with lipopolysaccharide (LPS) and nuclear receptor agonists in an IRAK-1 dependent fashion.

In addition to detecting binding of IRAK-1 to its substrates and/or the enzymatic activity of IRAK-1, the methods of the invention can identify interaction of IRAK-1 with its substrate(s) by way of gene expression of genes regulated by the substrate(s), such as by way of Northern blotting or RT-PCR of gene transcripts. Further, the enzymatic activity of IRAK-1 can be detected by assaying for production of proteins from regulated genes. Detection of such proteins can be by way of, for example, immunodetection. In addition, the enzymatic activity of IRAK-1 can be detected by in vitro or in vivo detection of the physiological effects of IRAK-1 activity. For example, production of foamy macrophage cells, activation of T-helper cells, or activation of T-regulator cells, can be detected.

Novel molecular targets of IRAK-1 include cellular proteins, such as Rac1 and NADPH oxidase. The targets also include transcription factors, such as C/EBPδ, which is positively activated by IRAK-1, and NFATc2, RARα, LXRα, PPARα, and PGC-1, which are suppressed by IRAK-1. Effector genes controlled by IRAK-1 include NOX1, MCP-1, iNOS, IL-6, and LCN2, which are positively induced, and ABCA1, Arginase 1, CPT-1, and MCAD, which are negatively suppressed.

Another aspect of the invention relates to cell-based assays. While cell-free in vitro assays are fully satisfactory for identification of substances that affect the activity of IRAK-1 and affect the interaction of IRAK-1 with its substrates, cell-based assays provide a better system for determining the in vivo activity of the substances. More specifically, cell-based assays include the additional complexity of intact cells in determining the effects of substances. The use of intact cells for assays allows the practitioner to gain a better understanding of the full effect of the substances on the physiology of cells. Cell-based assays thus provide a higher level of confidence in the predicted in vivo activity of the substances, and improve the drug discovery process. The cell-based assays can use normal cells or can use mutant cells, for example, cells that are deleted for IRAK-1.

The invention thus provides assays for identification of bioactive substances (e.g., drugs, lead compounds, etc.) that can be used for the treatment of diseases and disorders involving IRAK-1. In exemplary embodiments, the substances can be used to treat, either therapeutically or prophylactically, diseases and disorders involving inflammation, particularly inflammation resulting from IRAK-1 activity.

### BRIEF DESCRIPTION OF THE DRAWINGS

The accompanying drawings, which are incorporated in and constitute a part of this specification, illustrate several embodiments of the invention, and together with the written description, serve to explain certain principles of the invention.

FIG. 1 is a schematic generally showing the IRAK-1 and IRAK-1 ΔC proteins discussed in this disclosure.

FIG. 2 is a graph showing the effect of IRAK-1 on NFAT activity.

FIG. 3 is a protein gel stained for phosphorylated protein kinase B (P-Akt), showing the effect of IRAK-1 on P-Akt expression.

FIG. 4 depicts protein gels stained for IRAK in human brain samples taken from patients at different ages.

FIG. 5 depicts protein gels stained for phosphorylated Tau protein in samples comprising IRAK-1 and those lacking IRAK-1.

FIG. 6, Panels A-C, presents data supporting the role of a particular sequence of IRAK-1 in its interaction with a substrate, Rac1.

FIG. 7, Panels A-D, presents data showing that IRAK-1 is required for LPS-induced activation of Rac1 and generation of reactive oxygen species (ROS).

FIG. 8, Panels A and B, presents data showing that IRAK-1 is involved in LPS-induced C/EBPγ expression.

FIG. 9, Panels A-D, presents data showing that IRAK-1 suppresses activity of transcription factor RARα by suppressing nuclear translocation of RARα.

FIG. 10 presents data showing that IRAK-1 suppresses activity of transcription factor NFAT.

FIG. 11, Panels A and B, presents data supporting a molecular mechanism for IRAK-1 mediated regulation of NFAT.

FIG. 12, Panels A-C, presents data showing that the C-terminal region of IRAK-1 is involved in interaction with NFAT.

FIG. 13, Panels A-D, presents data showing that IRAK-1 suppresses transcription factor PPARα.

FIG. 14 presents data showing that IRAK-1 suppresses transcription factor LXRα.

FIG. 15, Panels A and B, presents data showing that IRAK-1 suppresses transcription factor PGC1.

FIG. 16, Panels A and B, presents data showing that IRAK-1 activates expression of MCP-1 and NOX1 in macrophages by inducing the expression of NOX1 via C/EBPδ.

FIG. 17 presents data showing that IRAK-1 induces the expression of MCP-1 via C/EBPδ.

FIG. 18, Panels A-E, presents data showing that IRAK-1 suppresses the expression of ABCA1 in macrophages.

FIG. 19, Panels A and B, presents data showing that IRAK-1 induces Th17 cells and suppresses Treg cells.

FIG. 20, Panels A and B, present data showing the involvement of IRAK-1 in production of IL-17 by Th17 helper cells in vivo.

FIG. 21, Panels A and B, present data showing that IRAK-1 plays a role in repressing Foxp3, and thus plays a role in developing T cells as T helper, rather than T regulator, cells.

FIG. 22 presents data showing that IRAK-1 is involved in development of atherosclerosis.

FIG. 23 presents data showing that IRAK-1 is activated in human leukocytes in atherosclerosis.

FIG. 24 presents data showing the correlation between certain IRAK-1 SNPs and cardiovascular disease.

FIG. 25 presents data showing that Tollip is an adaptor facilitating IRAK-1 function by way of interaction of the C2 domain of Tollip with PI3P.

FIG. 26 presents a schematic of IRAK-1 regulation of cellular molecules, leading to effects on inflammation.

FIG. 27 presents a schematic of IRAK-1 regulation of cellular molecules, leading to effects on macrophage physiology.

FIG. 28 presents a schematic of a dual-regulatory role for IRAK-1 in production of reactive oxygen species (ROS) as a component of the immune and inflammatory responses.

### DETAILED DESCRIPTION OF VARIOUS EMBODIMENTS OF THE INVENTION

Reference will now be made in detail to various exemplary embodiments of the invention, examples of which are illustrated in the accompanying drawings. The following detailed description is provide to give the reader a better understanding of certain details and features of the invention, and is not intended as a limitation on the scope or content of the invention or any of its various aspects.

In various embodiments of the invention, methods of affecting the biological and biochemical activity of substrates of IRAK-1 are discussed. In general, these substrates play roles in various diseases or disorders, whether as actual biochemical bases for the diseases and disorders, or merely as markers of the diseases and disorders. Indeed, the presence of certain forms, and in particular phosphorylation states, of certain proteins is indicative of a disease or disorder, and can be used not only as a marker for monitoring development and progression of the disease or disorder, but for detection and diagnosis as well.

Based on the intrinsic nature of IRAK-1 activity, it is envisioned according to the invention that IRAK-1 phosphorylates a family of potential protein substrates possessing at least one Serine/Proline-rich motif. So far, IRF-7 has been clearly defined as one of the substrates for IRAK-1. Intriguingly, IRF-7 possesses a Serine/Proline-rich motif. In addition, according to the invention, it is disclosed that several distinct molecules performing unique functions also have the Serine/Proline-rich motif. These molecules include NFATs (NFAT1, 2, 3, 4), VASP, IRS molecules (IRS-1, 2, 3, 4, 5, 6, 7), RARα, LXRα, Tau protein, small GTPase activating proteins, HIF1 alpha, IKK epsilon, and a phosphatase PHLPP. The invention provides direct experimental evidence indicating that IRAK-1 is responsible for phosphorylating NFAT molecules and Tau proteins. Indirect physiological data indicates that IRAK-1 is also involved in phosphorylating IRS-1 and PHLPP, and subsequently regulates Akt activity. Functional evidence indicates that C/EBPδ, RARα, LXRα, and PPARα serve as either direct or indirect downstream targets for IRAK-1. IRAK-1 contains a novel motif (LWPPPPSP; SEQ ID NO:2), which can interact with EVH-1 domain as well as the PH domain. While not being limited to any particular mechanism of action, this motif might help to bring IRAK-1 into close proximity with its substrates or binding partners. Furthermore, there are two stretches of the SSSS (SEQ ID NO:3) motif within the C-terminus of IRAK-1, which may serve to either regulate IRAK-1 activity or bind with its substrates.

According to the invention, IRAK-1 can be used to identify substances that can affect (either positively or negatively) the interaction of IRAK-1 with its substrates. The substances can be considered drug candidates for regulation of inflammation and diseases/disorders involving inflammation. It is to be understood that the use of the term IRAK-1 herein can include not only full-length IRAK-1, but portions of IRAK-1 having binding and/or phosphorylation activity. The LWPPPPSP motif discussed above is one such portion of IRAK-1 that can be used in assays. Likewise, it has been found that the C-terminal portion of IRAK-1, including at most residues 548-712, is involved in IRAK-1 interaction with NFAT. Thus, N-terminally truncated versions, or versions with deletions in the N-terminal region, can function in some assays in the same manner as full-length IRAK-1.

Through genotyping over 4000 patients suffering cardiovascular diseases (hypertension and atherosclerosis), it has now been discovered that several IRAK-1 single nucleotide polymorphisms (SNPs) are closely linked with higher risks for cardiovascular diseases. In order to determine the mechanism for IRAK-1 involvement, transcription factor reporter assays were performed to search for transcription factor(s) controlled by IRAK-1. It was discovered that IRAK-1 expression can significantly suppress NFAT reporter activity by phosphorylating and inactivating NFAT. Because elevated NFAT has been solidly linked with the pathogenesis of cardiovascular diseases, this finding uncovers the mechanistic connection between IRAK-1 gene variations and cardiovascular risks.

In one general aspect, the invention provides a method of affecting the phosphorylation state of a target protein involved in a disease or disorder. As used herein, the terms disease and disorder are to be interpreted in their broadest sense as used in the medical arts. They thus include diseases and disorders that are due to all mechanisms, including, but not necessarily limited to, those that have an intrinsic genetic basis (e.g., inherited, resulting from one or more mutations acquired during life) and those that are acquired as a result of external effects (e.g., through infectious agents, diet, stress, activity levels, aging). Non-limiting examples of certain diseases and disorders are discussed below with reference to the figures.

The method of affecting the phosphorylation state of a target protein comprises affecting the ability of an IRAK-1 protein or fragment thereof to contact the target protein. As used herein, the term IRAK-1 protein means any protein, polypeptide, or peptide having a sequence identical or similar to at least a portion of the sequence that can be found in GenBank under accession number P51617 (SEQ ID NO:1). An IRAK-1 protein according to the invention may thus be a human protein or a fragment of a human protein, or a non-human animal protein having identity to the human IRAK-1 protein. Identity levels can be of any level that allows for presumptive identification of the protein as an IRAK-1 protein. Preferably, identity levels are of at least 40% (using SEQ ID NO:1 as a basis for comparison), at least 50%, at least 60%, at least 70%, at least 80%, at least 90%, at least 95%, at least 98%, at least 99%, or about 100%. Where fragments are used, the fragments may have any level of identity to SEQ ID NO:1 as a whole, but preferably have high (e.g., greater than 50%) sequence identity to SEQ ID NO:1 over the length of the fragment. In some situations, total identity is not as important as conservation of one or more particular residues. In such situations, the presence of such residues will be sufficient to impart an activity to the fragment (e.g., kinase activity, ability to bind to a protein). Of course, due to the degeneracy of the genetic code, various codons can be used for each residue or a desired protein. Such variation is encompassed by the present invention.

According to the method, the amount of contact of the IRAK-1 and target protein is related to the amount of phosphorylation of the target protein. That is, according to the method, the target protein acts as a substrate for the IRAK-1, where an IRAK-1 having kinase activity phosphorylates the target protein and an IRAK-1 lacking kinase activity (e.g., a mutant form of the protein) does not phosphorylate, and in some situations blocks phosphorylation, of the target protein. Thus, in embodiments, the IRAK-1 protein has protein kinase activity for the target protein, and contact of the IRAK-1 protein results in phosphorylation of the target protein, which affects the target protein's activity (raises or lowers it). In other embodiments, the IRAK-1 protein does not have protein kinase activity for the target protein, and contact of the IRAK-1 protein with the target protein reduces phosphorylation of the target protein by other proteins. For example, binding of a kinase-inactive IRAK-1 protein, polypeptide, or peptide to the target protein can essentially titrate out the target protein, lowering the number of target proteins available for phosphorylation by kinase-active IRAK-1 proteins. The net effect in a population of target proteins is a lower phosphorylation state of the population.

Certain diseases and disorders involve undesirable phosphorylation of one or more substances in cells, and particularly undesirable phosphorylation of proteins, such as those involved in transcription regulation pathways, responsiveness to external stimuli, and cell growth and maintenance. The present invention recognizes, for the first time, the role of IRAK-1 in many of these processes, and recognizes the usefulness of IRAK-1, its fragments, and its inhibitors, in treating or preventing diseases and disorders. For example, diseases and disorders involving NFAT family members, PH domain-containing proteins, and neurodegenerative proteins, such as Tau protein, may be treated by altering the phosphorylation states of these proteins using IRAK-1 and its inhibitors.

Accordingly, in an aspect of the invention, a method of treating a patient having or being susceptible to developing a disease or disorder involving IRAK-1 kinase activity is provided. In general, the method comprises contacting a cell comprising a protein that has an activity that is regulated by IRAK-1 with a substance that alters the level or activity of the protein as a result of phosphorylation by IRAK-1, wherein the altered protein activity results in a detectable change in at least one clinical symptom of the disease or disorder or reduces or prevents the likelihood of development of at least one clinical symptom of the disease or disorder. As used herein, the term “contacting” means any action that results in physical contact of substances of interest. Thus, contacting can be an action that directly causes two or more substances to come into contact or that indirectly causes two or more substances to come into contact. For example, contacting can comprise exposing two or more substances to each other in an environment that is suitable for contact of the substances for an amount of time that is sufficient for contact to occur. It thus may included adding a composition comprising a first substance (e.g., an IRAK-1 protein) to a liquid composition comprising a second substance (e.g., a culture medium comprising cells containing an IRAK-1 substrate) and allowing adequate time for the first substance to diffuse through the liquid and contact the second substance. In the context of in vivo treatment of a disease or disorder, contacting may comprise administering a sufficient amount of a substance to allow that substance to contact a cell of interest and exert an effect, typically as a result of being taken into the cell. Those of skill in the art of medicine are capable of devising appropriate dosing regimens to achieve this result.

According to one embodiment of the invention, an ex vivo method of treatment is provided. Specifically, one or more cells or cell types involved in the inflammation process can be removed from a patient, and the cells treated with a substance that affects the activity of IRAK-1. The treated cells will then have an altered protein profile, which has a reduced or no ability to promote inflammation. The altered cell is then reintroduced into the patient to effect a treatment for inflammation. While the treatment might be transient, it still can ameliorate some or all of the deleterious effects of the inflammation to be treated. Alternatively, cells can be removed from a patient and can be genetically modified to knock out IRAK-1 expression. The genetically modified cells can then be reintroduced into the patient and reduce inflammation and treat diseases and disorders associated with inflammation. In such embodiments, the invention represents a cell-based treatment method.

The present method includes both therapeutic and prophylactic treatment. In other words, patients already affected by a disease or disorder may be treated by the method (and other methods, discussed below) to cause a detectable change in the disease or disorder. Preferably, the change is a reduction or elimination of one or more clinical symptoms of the disease or disorder. More preferably, the change is elimination or cessation of one or more clinical symptoms. Most preferably, the change is elimination of the disease or disorder, which may be permanent and require no further treatment or may be ephemeral or permanent and require continued treatment to remain at the state achieved. When used prophylactically, the method may be used to treat individuals to stop or delay development of one or more clinical symptoms of a disease or disorder. Prophylactic treatment will typically be performed on subjects suspected of having a sub-clinical state of a disease or disorder or on subjects suspected of being susceptible to a disease or disorder. For example, in the context of treating diabetes or related complications, the method may be prophylactically performed on individuals with a family history of diabetes. Likewise, for example, within the context of treating neurodegenerative diseases, the method may be practice on elderly patients, such as those 75 years or older. It also could be practiced, for example, on patients having neurodegenerative disorders that show early onset, such as Parkinson's disease (e.g., onset in patients in their 20s, 30s, 40s, 50s, or 60s). The age of onset or clinical detection is not critical, and the invention relates to all diseases and disorders involving IRAK-1 and its substrates.

According to the method, the substance that alters the level or activity of the protein as a result of phosphorylation by IRAK-1 can cause an increase in phosphorylation due to IRAK-1 or a decrease in phosphorylation due to IRAK-1. It has been found that IRAK-1 exerts its effects on cellular proteins by phosphorylating the proteins. The effects of the phosphorylation vary depending on the substrate: some substrates are inactivated by phosphorylation whereas others are activated (the “activated” being the state involved in disease development and/or progression).

The substance that alters the level of activity of a protein may be IRAK-1 or a fragment thereof having the desired activity. As discussed above, the IRAK-1 may be a full-length animal protein, a fragment, truncated, or otherwise mutated version of IRAK-1, or a molecule having appropriate spacing of residues that have a desired activity. The substance may be obtained or produced in any suitable fashion, including but not limited to total chemical synthesis, recombinant synthesis in vitro or in vivo, and combinations thereof.

Alternatively, the substance may be a substance that affects the activity or expression of IRAK-1. Among the non-limiting examples of such substances, mention can be made of Pam3CSK4 (Triacylated Cys-Ser-Lys-Lys-Lys-Lys; SEQ ID NO:4), Lipid A, Poly(I:C), and flagellin, all of which can activate IRAK-1. Furthermore, the molecule may be, for example, an antibody that binds IRAK-1 and reduces or blocks its ability to bind or phosphorylate a substrate.

In embodiments, the target protein having its activity altered is an NFAT family member, a protein having a PH motif, or a Tau protein. It has now been found that substrates for IRAK-1 include, but are not necessarily limited to, NFAT family members, proteins comprising a PH domain, and Tau protein. Other substrates are discussed below.

In another aspect, the invention provides a method of phosphorylating or blocking phosphorylation of one or more NFAT family member proteins. Depending on the purpose and goal, the method may be practiced in vitro or ex vivo, for example in a cell-free system or in a culture media comprising cells in culture, or practiced in vivo as a treatment method. In general, the method comprises exposing one or more NFAT member proteins to an IRAK-1 protein under conditions that allow contact of the IRAK-1 protein and the NFAT protein, resulting in phosphorylation of the NFAT protein by the IRAK-1 protein or blocking of phosphorylation. In cell-free systems, exposing for an adequate amount of time is typically sufficient to cause contact of the two substances. However, in systems that involve uptake of one or both of the substances, the cells or the substances may be treated to improve uptake. For example, cells may be treated with one or more other substances or phenomena (e.g., heat, cold) or the substances may be combined with carriers to facilitate movement across a cellular wall or membrane.

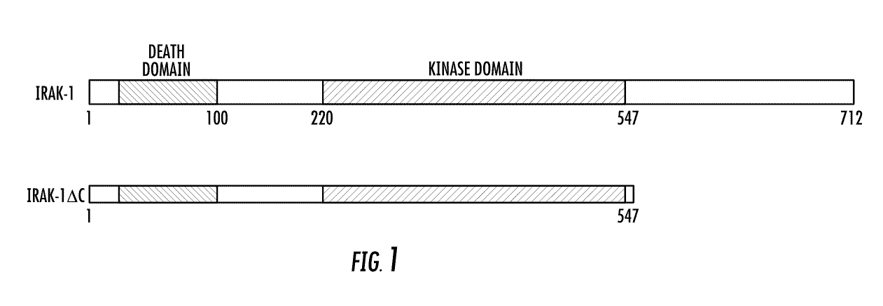

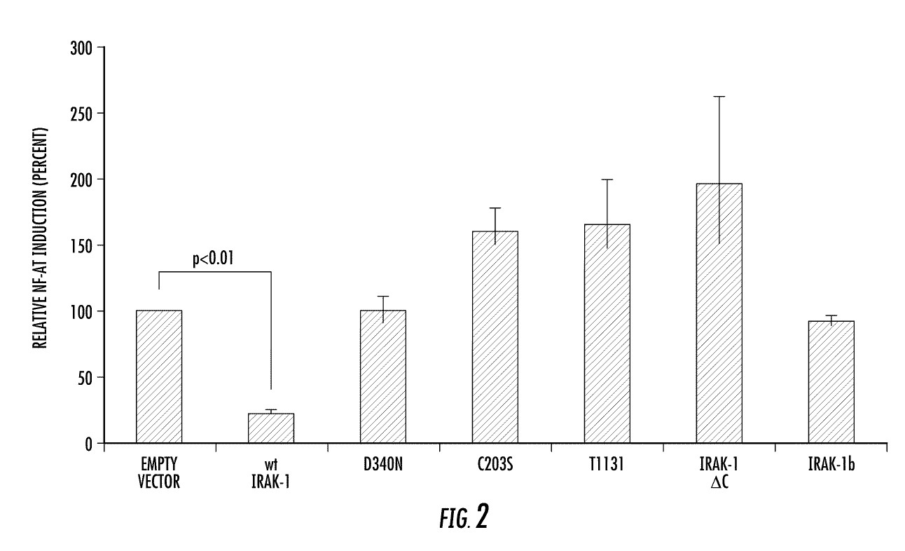

In embodiments, the IRAK-1 protein is a polypeptide or peptide fragment of a full-length IRAK-1 protein, which has NFAT protein kinase activity (e.g., contains residues 1-about 521 of an IRAK-1 protein). In embodiments, the fragment comprises residues 220-547 of IRAK-1, residues 100-547 of IRAK-1, resides 20-547 of IRAK-1, or any fragment encompassed by these ranges. Various domains and features of IRAK-1 and a C-terminally truncated version used in the Examples below are depicted in FIG. 1. It is important to note at this point that, where a value is stated herein, unless otherwise specifically noted, the value is not meant to be precisely limited to that particular value. Rather, it is meant to indicate the stated value and any statistically insignificant values surrounding it. As a general rule, unless otherwise noted or evident from the context of the disclosure or from the nature of experiments and their associated intrinsic variance, each value includes an inherent range of 5% above and below the stated value. At times, this concept is captured by use of the term “about”. However, the absence of the term “about” in reference to a number does not indicate that the value is meant to mean “precisely” or “exactly”. Rather, it is only when the terms “precisely” or “exactly” (or another term clearly indicating precision) are used is one to understand that a value is so limited. In such cases, the stated value will be defined by the normal rules of rounding based on significant digits recited. It is further to be understood that, where a range of values are given, every particular value within that range is encompassed by the range, without the need for each particular value to be specifically recited.

In embodiments, the NFAT protein family member is NFAT-1, NFAT-2, NFAT-3, or NFAT-4.

In embodiments, the method comprises altering the ratio of un-phosphorylated to phosphorylated NFAT protein or other IRAK-1 substrate in a cell. In general, the method comprises contacting the cell with a substance that causes or blocks phosphorylation of a substrate under conditions that allow for the substance to enter the cell and cause, directly or indirectly, phosphorylation of at least one substrate. For example, the method may comprise exposing a cell to IRAK-1 or a portion thereof under conditions that allow for uptake of the IRAK-1 or portion thereof and allow for contact, within the cell, of the IRAK-1 or fragment and an NFAT protein, resulting in phosphorylation or dephosphorylation of the NFAT protein. In embodiments, the method is a method of phosphorylating an NFAT protein. In other embodiments, the method is a method of reducing the amount of un-phosphorylated NFAT in a cell. In other embodiments, the method is a method of reducing the activity of one or more NFAT proteins in a cell. The method can be practiced both in vitro and in vivo and, as mentioned above, can be practiced on other IRAK-1 substrates.

In another aspect, the invention provides a method of treating a patient having a disease or disorder involving the activity of an NFAT family member. In general, the method comprises exposing at least one cell of the patient to a substance that alters (reduces or increases) the amount of an un-phosphorylated NFAT family member protein in the cell by causing phosphorylation or dephosphorylation of NFAT proteins in the cell, wherein phosphorylation or dephosphorylation results in a change in the activity of the NFAT protein. In embodiments, the method comprises administering to the patient an amount of IRAK-1 protein that is sufficient to reduce the amount of un-phosphorylated NFAT protein to a level that results in a change in a detectable clinical symptom of the disease or disorder. As with other aspects, the IRAK-1 protein may be a full-length protein or a fragment thereof having protein kinase activity, and preferably NFAT protein kinase activity. Likewise, the NFAT protein may be any NFAT family member protein. Further, the method may be therapeutic or prophylactic.

In embodiments, the methods of the invention relating to altering the phosphorylation state of NFAT can be methods for treatment of cardiovascular diseases and disorders. In general, the method can comprise contacting at least one cell containing a protein of the NFAT family with a substance that will alter the amount of un-phosphorylated NFAT protein in the cell by phosphorylating or dephosphorylating the NFAT protein. Phosphorylating the NFAT family protein can be accomplished by interaction with IRAK-1 or a portion of it having NFAT-phosphorylating activity (e.g., some or all of the residues from 1 to about 547). The present invention discloses, for the first time, that IRAK-1 (Interleukin-1 Receptor Associated Kinase-1, also known as IRAK, IRAK1, Genbank Accession: NP_{—}001560) is a protein kinase involved in regulation of the activities of several transcription factors contributing to the pathogenesis of heart hypertrophy, hypertension, and atherosclerosis. The transcription factors include NFATs (including NFAT1, 2, 3, and 4). Proper IRAK-1 function is involved in regulation of NFATs to prevent the pathogenesis of these cardiovascular diseases, as well as other complications, such as diabetes. However, proper IRAK-1 function has now been linked to development of diseases and disorders, and the present invention contemplates interfering with the proper or normal function of IRAK-1 as an intervention for diseases and disorders.

Furthermore, the present invention discloses that there are several regions in IRAK-1 that are involved in regulating the activities of certain transcription factors. In particular, there is a region in the C-terminus (covering amino acids 548-712) of IRAK-1 that is involved, if not necessary, for IRAK-1 interaction with NFATs. Small molecules or other small or large compounds, either chemical or biological, that target these regions can disrupt the interaction between IRAK-1 and these transcription factors. These small molecules or compounds can serve as therapeutic reagents to treat human cardiovascular diseases including hypertension, atherosclerosis and other complications.

In yet a further aspect, the invention provides a method of reducing or blocking phosphorylation of one or more proteins having a Pleckstrin Homology (PH) domain. In general, the method comprises exposing one or more PH domain-containing proteins to an IRAK-1 protein lacking protein kinase activity, a fragment thereof lacking protein kinase activity, or in the presence of a substance that inhibits or blocks contact of the IRAK-1 protein and the PH domain-containing protein under conditions that allow contact of the IRAK-1 protein and the PH domain-containing protein, resulting in a reduction or abolition of phosphorylation of the PH domain-containing protein by the IRAK-1 protein. In embodiments, the IRAK-1 protein is a polypeptide or peptide fragment of a full-length IRAK-1 protein, which is not capable of binding to the target PH domain-containing protein. In other embodiments, the IRAK-1 protein is a polypeptide or peptide fragment of a full-length IRAK-1 protein, which is capable of binding to the target PH domain-containing protein, but not capable of phosphorylating the target protein. In yet other embodiments, the method comprises exposing the IRAK-1 protein, the target PH domain-containing protein, or both, to a substance that interferes with contact between the IRAK-1 protein and the PH domain-containing protein.

The present invention, for the first time, identifies IRAK-1 as a protein kinase involved in regulating the activities of several signaling proteins involved in insulin resistance. Insulin resistance is the key mechanism underlying human diabetes and related complications. This discovery is a key piece in solving the puzzle of insulin resistance and preventing insulin resistance and in curing type II diabetes. The present invention discloses a unique region on IRAK-1 that is involved in regulating insulin signaling. This region, covering amino acid 162 to amino acid 176 (PSPASLWPPPPSPAP; SEQ ID NO:5) is involved in IRAK-1 interaction and regulation of PH (Pleckstrin-Homology) domain-containing proteins, such as PDK-1, Akt/PKB, IRS (insulin receptor substrate) proteins, and small GTPase activating proteins. This region is also involved in binding with VASP and other EVH-1 domain containing proteins. A small region, LWPPPP (SEQ ID NO:6) is also implicated in the interaction, as are sequences surrounding and/or encompassing this sequence that include the tryptophan (W) residue.

Binding of IRAK-1 with these PH domain-containing proteins regulate their activities and modulate their activations. Activation of Akt/PKB and IRS proteins are involved in insulin signaling and subsequent metabolic processes, including glucose transport and turnover, as well as cell growth. Because IRAK-1 activity modulates PDK-1, Akt/PKB, IRS, and other PH-domain containing proteins, malfunction or overactivity of IRAK-1 contributes to insulin resistance and diabetes. Small molecules or other compounds targeting the above-described IRAK-1 region can be used to intervene with the functions of the PDK-1, Akt/PKB, and IRS proteins. This targeting can facilitate Akt/PKB and IRS mediated downstream signaling and insulin response, which in turn will prevent the pathogenesis of human diabetes.

The present invention relates to all PH domain-containing proteins that are involved in diseases and disorders. Thus, in embodiments, the PH domain-containing protein is PDK-1, PKB, IRS, or a small GTPase activating protein. In embodiments, the PH domain-containing protein is involved in regulation or development of diabetes.

Although in some embodiments the inhibitor is a small molecule, in some other embodiments, the inhibitor is a fragment of IRAK-1. That is, IRAK-1 fragments may be used to titrate out PH domain-containing proteins, thus reducing the number of phosphorylated PH domain-containing proteins in a cell, and reducing insulin insensitivity. As with other methods of the invention, the method may be practiced in vitro, for example in a cell-free system or in a controlled laboratory environment (e.g., tissue culture plate, microtiter plate), or in vivo, for example in an animal subject (also referred to herein as a patient, person, or animal).

In yet another aspect, the invention provides a method of treating a patient having or susceptible to developing a disease or disorder involving the activity of a PH domain-containing protein. In general, the method comprises exposing at least one cell of the patient to a substance that reduces the amount of a phosphorylated target PH domain-containing protein in the cell by reducing or eliminating phosphorylated forms of the target protein by reducing or blocking the interaction of IRAK-1 on the target protein. Reducing or blocking the interaction reduces the amount of phosphorylated PH domain-containing protein, and reduces or eliminates the disease or disorder, a detectable clinical symptom, or the likelihood of development. In embodiments, the method comprises administering to the patient an amount of a small molecule inhibitor (e.g., a drug or pharmaceutical) that interferes with interaction of an IRAK-1 protein and a PH domain-containing protein. In other embodiments, the method comprises administering to the patient an amount of an IRAK-1 protein or fragment thereof lacking kinase activity on the target PH domain-containing protein that is sufficient to reduce the amount of phosphorylated target protein to a level that results in a change in a detectable clinical symptom of the disease or disorder. In other embodiments, the method comprises administering to the patient an amount of a PH domain-containing protein or fragment thereof that is sufficient to bind to an IRAK-1 protein and reduce the amount of phosphorylated target PH domain-containing protein to a level that results in a change in a detectable clinical symptom of the disease or disorder. As with other aspects, the PH domain-containing protein may be a full-length protein or a fragment thereof having the ability to bind to IRAK-1. The PH domain-containing protein may be any such protein, including, but not limited to those involved in response to insulin. For example, the protein may be Akt, an IRS family member (e.g., IRS1, IRS2, IRS3, IRS4, IRS5, IRS6, IRS7), PDK, or FGD. As with other methods of treating, this method can include ex vivo treatment of cells, then reintroduction into the patient. Further, the method may be therapeutic or prophylactic.

In yet an additional aspect, the invention provides a method of reducing or blocking phosphorylation of a Tau protein, such as one found in the brain (e.g., in a neuron). In general, the method comprises exposing a Tau protein to an IRAK-1 protein lacking protein kinase activity, a fragment thereof lacking protein kinase activity, or in the presence of a substance that inhibits or blocks contact of the IRAK-1 protein and the Tau protein under conditions that allow contact of the IRAK-1 protein and the Tau protein, resulting in a reduction or abolition of phosphorylation of the Tau protein by the IRAK-1 protein. In embodiments, the IRAK-1 protein is a polypeptide or peptide fragment of a full-length IRAK-1 protein, which is not capable of binding to the target Tau protein. In other embodiments, the IRAK-1 protein is a polypeptide or peptide fragment of a full-length IRAK-1 protein, which is capable of binding to the target Tau protein, but not capable of phosphorylating the target protein. In yet other embodiments, the method comprises exposing the IRAK-1 protein, the target Tau protein, or both, to a substance that interferes with contact between the IRAK-1 protein and the Tau protein (e.g., a drug). In embodiments, the Tau protein is involved in regulation or development of a neurological disease, such as but not limited to Alzheimer's Disease (AD) and Parkinson's Disease. In embodiments, the inhibitor is a fragment of IRAK-1. The method may be practiced in vitro, for example in a cell-free system or in a controlled laboratory environment (e.g., tissue culture plate, microtiter plate), or in vivo, for example in an animal subject. As with other methods of treating, this method can include ex vivo treatment of cells, then reintroduction into the patient.

The present invention discloses for the first time that IRAK-1 is involved in regulation of the phosphorylation state of Tau, which is a key protein involved in the pathogenesis of various neurological diseases, including Alzheimer's disease and Parkinson's disease. Tau protein is involved in the assembly of microtubules and related cytoskeleton structures. In particular, Tau protein plays a key role in maintaining proper neuronal cell structure and function. Hyper-phosphorylation of Tau in neuronal cells leads to the formation of neurofibillary tangles, Tauopathy, and related neurological diseases, including Alzheimer's disease and Parkinson's disease. It has now been discovered that IRAK-1 contributes to the phosphorylation of Tau protein. Furthermore, it has been discovered that full-length functional IRAK-1 is only present in aged human brain tissues, and is not present in young human brain tissues. Elevated IRAK-1 and its activity in aged human brains therefore contributes to the pathogenesis of various neurological diseases accompanied with the aging process, including Alzheimer's and Parkinson's. Among the advances provided by these discoveries are the ability to treat these diseases with inhibitors of IRAK-1-dependent Tau phosphorylation (e.g., IRAK-1 derived peptides that bind Tau but do not phosphorylate it, and small molecules or compounds that can disrupt the interaction between IRAK-1 and Tau). Data indicates that the C-terminus of IRAK-1 is involved in interaction with Tau protein.

In another aspect, the invention provides a method of treating a patient having or susceptible to developing a disease or disorder involving the activity of a Tau protein. In general, the method comprises exposing at least one cell of the patient to a substance that reduces the amount of a phosphorylated target Tau protein in the cell by reducing or eliminating phosphorylated forms of the target protein by reducing or blocking the interaction of IRAK-1 on the target protein. Preferably, the cell is a brain cell, such as a neuron. Reducing or blocking the interaction reduces the amount of phosphorylated Tau protein, and reduces or eliminates the disease or disorder, a detectable clinical symptom, or the likelihood of development. In embodiments, the method comprises administering to the patient an amount of an IRAK-1 protein or fragment thereof lacking kinase activity on the target Tau protein that is sufficient to reduce the amount of phosphorylated target protein to a level that results in a change in a detectable clinical symptom of the disease or disorder. As with other aspects, the IRAK-1 protein may be a full-length protein or a fragment thereof lacking protein kinase activity. In other embodiments, the method comprises administering to the patient an amount of a Tau protein or fragment thereof that is sufficient to bind to an IRAK-1 protein and reduce the amount of phosphorylated target Tau protein to a level that results in a change in a detectable clinical symptom of the disease or disorder. The Tau protein may be a full-length protein or a fragment thereof having the ability to bind to IRAK-1; however, to improve availability in brain tissue, the protein is preferably relatively short, such as a peptide having fewer than 100 residues. Further, the method may be therapeutic or prophylactic.

In a further aspect, the invention provides a method of identifying compounds that affect the interaction of IRAK-1 with one or more of its substrates. In general, the method comprises exposing an IRAK-1 protein having one or more known activities to one or more substances under conditions where the IRAK-1 and substance can come into contact; and determining the effect of the substance(s) on an activity of the IRAK-1. Among the various activities of IRAK-1 include, but are not necessarily limited to: binding to a substrate, phosphorylating a substrate, and blocking phosphorylation of a substrate by a kinase. Thus, for example, the method can comprise exposing an NFAT protein or another substrate discussed herein to one or more small molecules, exposing the substrate-small molecule mixture to IRAK-1, and determining whether phosphorylation of the substrate can be altered by the small compounds. While small molecules are exemplified herein, it is to be understood that the invention contemplates all substances that have the described activities. The step of determining can be any action that can reasonably indicate that the status of the substrate has been altered. It thus can be the act of directly detecting interaction of IRAK-1 with its substrate, for example by co-precipitation of the two molecules. Alternatively, it can be the act of indirectly detecting interaction, for example by determining the phosphorylation state of the substrate after (and preferably before as well) combining the IRAK-1 and its substrate. Yet again, it can be by way of indirect detection of interaction, such as by assaying for expression of an RNA transcript or gene expression product of a gene known to be transcribed under the control of an activated factor. In addition, it can be by way of indirect detection of interaction, such as by determining the differentiation state of an immune cell, such as a T cell or macrophage. Likewise, it can be by way of indirect detection of interaction by detection of the production or reactive oxygen species.

In another non-limiting example of the screening method, the method can comprise combining an unphosphorylated Tau protein, an IRAK-1 protein, and one or more substances, and determining if the Tau protein becomes phosphorylated. Likewise, a similar screening method may be practiced using a PH domain-containing protein. Similar screening methods can be practiced using the other IRAK-1 binding partners discussed herein.

The order of addition of substances is not critical to practice of the screening method. Thus, IRAK-1 and its substrate protein may be bound initially, then subjected to one or more substances, which might replace IRAK-1 in binding to the substrate. Where multiple substances are screened in a single iteration of the method, the method may be performed again, with all or fewer than all of the originally-present substances to confirm the original results and/or to eliminate certain substances as potential active substances. In preferred embodiments, the method is repeated with fewer and fewer substances per sample until few or a single substance is identified as having activity. Of course, as with any method for identifying a substance, one or more positive and negative control reactions may be performed to confirm results and ensure that reactions and steps were accomplished as desired.

While the screening method may be practiced both in vivo and in vitro, it is preferred that the method be practiced in vitro, which includes cell-based assays. An in vitro assay is typically faster and more reproducible, and thus can process more substances than an in vivo assay. However, where a substance of interest is identified, the method may be practiced in vivo to determine if the in vitro results can be reproduced in vivo or to optimize concentrations or other parameters. In some embodiments, the method is a high-throughput screening method to screen a library of compounds, such as a random library of chemical structures.

In yet another aspect, the invention provides a composition. In general, the composition is useful for performing a method according to at least one aspect of the invention. For example, the composition may comprise an isolated or purified (at least to some extent) IRAK-1 protein or fragment thereof having protein kinase activity. Alternatively, it may comprise an isolated or purified IRAK-1 protein lacking kinase activity for a particular target protein. Likewise, it might be a full-length, fragment, or otherwise mutant form of an IRAK-1 substrate protein, which can be used to titrate the activity of an IRAK-1 protein in vitro or in vivo. While not required, it is preferred that the IRAK-1 protein or fragment have the ability to at least bind to a target substrate protein in a manner sufficient for detection of the binding.

Typically, the composition comprises a protein as described above and at least one other substance that is biologically tolerable or is suitable for use in an in vitro assay for IRAK-1 activity. Preferably, the IRAK-1 protein or fragment is purified or isolated from other components of a cell from which it is derived to an extent at least that the other components do not interfere significantly with an assay for IRAK-1 activity, such as protein binding or protein kinase activity. In embodiments, it is purified to substantial purity. That is, in these embodiments and using standard detection techniques, the IRAK-1 protein or fragment is found to be present as the only proteinaceous substance. The IRAK-1 can also be found in a less purified form, such as a form making up 99% or more, 98% or more, 95% or more, or 90% or more of the proteinaceous material in the sample. Lower levels of purity are also encompassed; however, they are less preferred.

In embodiments, the composition comprises a fragment or other mutant form of full-length IRAK-1 (e.g., SEQ ID NO:1). In some embodiments, the IRAK-1 protein comprises a substrate-binding activity, such as the ability to bind an NFAT protein family member in vitro. In some embodiments, the IRAK-1 protein comprises a protein kinase activity, such as the ability to phosphorylate an NFAT family member protein. In some embodiments, the IRAK-1 protein does not comprise protein kinase activity, but preferably retains substrate binding ability (e.g., it can bind to a Tau protein, but not phosphorylate it).

In some embodiments, typically those relating to screening for a drug, the composition comprises an IRAK-1 protein and a substance that can interfere with an activity of IRAK-1. For example, the composition may comprise IRAK-1 and a substance that blocks IRAK-1 from phosphorylating a substrate.

In embodiments relating generally to cell culture testing or to in vivo use of IRAK-1 proteins, typically, the composition comprises sufficient IRAK-1 protein or fragment to enter a target cell when exposed to the cell. In embodiments, the composition is a pharmaceutical composition suitable for administration to subjects in need thereof, such as one comprising IRAK-1 in an amount sufficient to treat a subject. It is typically found in the presence of one or more pharmaceutically acceptable excipients, carriers, buffers, salts, and the like. The compositions are often aqueous, for use in administration by injection, infusion, orally, or mucosally. However, the compositions may be solid for use in administration by oral route or for storage until reconstitution with a liquid, such as an aqueous liquid. In some embodiments, it is preferred that the protein or fragment is incapable of traversing the blood-brain-barrier, whereas in other embodiments, it is preferred that the protein or fragment is capable of doing so. The IRAK-1 protein may be modified as needed to improve its ability to cross the BBB or to resist crossing the BBB.

Flowing logically from the above description of aspects and embodiments, it is evident that the invention encompasses use of IRAK-1, a portion of IRAK-1 including the LWPPPPSP peptide, the C-terminus fraction, or any other dominant negative version of IRAK-1, its substrates, and substances that bind or otherwise interact with them, in therapeutic and diagnostic applications. Thus, it should be evident that the invention, in an aspect, relates to methods of diagnosing a disease or disorder involving IRAK-1. In general, the methods comprise detecting the presence or activity of IRAK-1 (or a fragment thereof having kinase activity) and correlating that presence or activity with one or more diseases or disorders. In general, the presence of a particular protein or protein fragment is indicative of a disease or disorder, or the predisposition to develop a disease or disorder. The method can be practiced on a subject having clinical symptoms of a disease or disorder, such as diabetes or a neurological disorder. Alternatively, it can be practiced on a subject not showing any clinical symptoms of a disease or disorder, but suspected of, or at a risk of, developing a disease or disorder of interest. The disease or disorder may be any of those discussed herein, and the target for detection may likewise be any of the various proteins, peptides, or other substances discussed herein. In exemplary embodiments, the disease or disorder is inflammation or a disease or disorder associated with inflammation. Detection can be based on detection of a physical entity (e.g., direct detection of a protein by way of a protein gel), or based on detection of a biochemical activity (e.g., indirect detection of a protein by way of enzymatic activity or expression of a gene product). The assay or method can be practiced in vitro, in vivo, or as a combination of steps comprising the two. Typically, the method is performed in vitro or at least partially in vitro.

For example, the method may comprise detecting full-length IRAK-1, a C-terminally truncated form of IRAK-1, a fragment of IRAK-1 having substrate binding activity or protein kinase activity, or a mutant form that lacks substrate binding ability or protein kinase activity. Alternatively, the method may comprise detecting a substrate of IRAK-1, in either its phosphorylated or unphosphorylated form, the particular phosphorylation state being associated with a particular disease or disorder. Typically, phosphorylation state is determined as a function of the portion of molecule in a population. It is thus a relative measurement and will vary from system to system and from substrate to substrate. Those of skill in the art are fully capable of recognizing appropriate phosphorylation states to draw useful conclusions, particularly when control reactions are also performed. Fragments of the substrate comprising phosphorylation sites or binding sites for IRAK-1 may also be detected and correlated to diseases or disorders. For example, fragments comprising proline-serine rich sequences can be used. Detection can be by any suitable method, such as by way of use of antibodies that specifically bind certain peptide sequences, such as phosphorylated sequences or unphosphorylated sequences, by way of use of protein gels showing the presence of a protein or peptide of suitable size, by chromatography methods (e.g., affinity chromatography), by enzymatic assays, etc.

The present invention provides key information relating to the molecular mechanisms involved in the complex system of inflammation. The present invention, for the first time, recognizes IRAK-1 as a critical regulator of many of the proteins involved in the inflammation process. IRAK-1 is recognized herein as a regulator of not only intracellular processes that lead to production of molecules involved in inflammation, but also the activation of various immune cells that play important roles in the immune response in general and inflammation in particular. For example, the present invention provides for the regulation of the activity of macrophages: IRAK-1 favors the expression of pro-inflammatory cytokines, chomokines, and reactive oxygen species in macrophages; IRAK-1 suppresses the expression of anti-inflammatory mediators such as Arginase 1; and IRAK-1 suppresses the expression of ABCA1, and thus favors the formation of foam cells. Furthermore, the present invention provides for the regulation of the activity of T cells: IRAK-1 favors the differentiation of CD4Th 17 cells; and IRAK-1 suppresses the differentiation of CD4 T regulatory (Treg) cells. In addition, the present invention provides for the regulation of metabolic cells (e.g., mesangial cells, muscle cells, hypatocytes): IRAK-1 favors the expression of glucose metabolic genes; IRAK-1 suppresses the expression of free fatty acid oxidation genes (e.g., CPT-1, MCAD); consequently, IRAK-1 suppresses fatty acid oxidation in metabolic cells.

Flowing logically from the above description of aspects and embodiments, it is evident that the invention encompasses use of dominant negative IRAK-1, a fraction of IRAK-1 that blocks IRAK-1 activity or its interaction with its downstream targets (Rac1, NFAT, C/EBPβ/δ, STAT3, RARα, LXRα, PPARα), its substrates, and substances that bind or otherwise interact with them, in therapeutic and diagnostic applications. In embodiments, methods of drug discovery are provided that assay for production, alteration, or activity of downstream targets of IRAK-1, including STAT3, C/EBPδ, NFAT, NFκB, RARα, LXRα, PPARα, and PGC-1. In embodiments, molecules that affect interaction of IRAK-1 with NFκB are not assayed.

The present invention provides a role for IRAK-1 in the differentiation of macrophages. In particular, IRAK-1 favors the differentiation of pro-inflammatory macrophages expressing reactive oxygen species, cytokines and chemokines (MCP-1, IL-6). IRAK-1 suppresses nuclear receptor mediated expression of anti-inflammatory mediators such as arginase 1 and ABCA1. The invention encompasses use of dominant negative IRAK-1, compounds that selectively blocks IRAK-1 interaction with the above mentioned substrates or inhibits IRAK-1 function towards these molecules targeting macrophages in vivo or in vitro, in therapeutic and diagnostic applications to treat inflammatory diseases, such as atherosclerosis, lupus, sepsis, and neuro-inflammation. The invention also encompasses in vitro screening of compounds that can specifically block IRAK-1 function towards macrophage activation in cultured macrophages and monocytes.

The present invention provides a role for IRAK-1 in the differentiation of T helper cells. In particular, IRAK-1 favors the differentiation of Th17 cells, and suppresses the differentiation of T regulatory cells. The invention encompasses use of dominant negative IRAK-1, compounds that selectively block IRAK-1 interaction with the above mentioned substrates or inhibit IRAK-1 function towards these molecules specifically targeting at T lymphocytes in vivo or in vitro, in therapeutic and diagnostic applications to treat inflammatory diseases such as atherosclerosis, lupus, sepsis, and auto-immune diseases. The invention also encompasses in vitro screening of compounds that can specifically block IRAK-1 function towards T-helper cell differentiation in cultured T lymphocytes.

The present invention provides a role for IRAK-1 in the regulation of fatty acid metabolism in metabolic cells and tissues including liver, kidney, and mesangial cells. In particular, IRAK-1 suppresses fatty acid oxidation, by suppressing the function of nuclear receptors including PPARα, RARα, and LXRs. The invention encompasses use of dominant negative IRAK-1, compounds that selectively blocks IRAK-1 interaction with the above mentioned substrates or inhibits IRAK-1 function towards these molecules in metabolic tissues and cells in vivo or in vitro, in therapeutic and diagnostic applications to treat inflammatory diseases such as atherosclerosis, lupus, sepsis, and auto-immune diseases. The invention also encompasses in vitro screening of compounds that can specifically block IRAK-1 function towards fatty acid oxidation in cultured metabolic cells including hepatocytes, mesnagial cells, and muscle cells.

The present invention also provides a role for IRAK-1 in vascular tissues: IRAK-1 favors foam cell formation; IRAK-1 favors infiltration of immune cells to inflammatory sites; and IRAK-1 increases tissue oxidative damage. Likewise, the invention provides a role for IRAK-1 in kidney, liver, and other organs and tissues: IRAK-1 suppresses fatty acid oxidation and decreases energy supplies. In addition, it is disclosed herein that IRAK-1 increase tissue oxidative damage. In peripheral lymph nodes, IRAK-1 increases Th17 cells. The present invention also shows that IRAK-1 facilitates the pathogenesis of atherosclerosis. Consequently, targeted inhibition of IRAK-1 with selective downstream molecular targets can be used to treat atherosclerosis. Also disclosed herein is the fact that IRAK-1 facilitates the pathogenesis of lupus. Consequently, targeted inhibition of IRAK-1 with selective downstream molecular targets can be used to treat lupus. IRAK-1 also is disclosed herein to contribute to neuro-inflammation.

The downstream function of IRAK-1 is also disclosed herein. Specifically, IRAK-1 is involved in TLR pathway-induced activation of the small GTPase Rac-1. Consequently, IRAK-1 is necessary for TLR-agonist induced generation of reactive oxygen species. IRAK-1 activates Rac-1 by directly associating with Rac-1, through the LWPPPPSP motif on IRAK-1. The therapeutic benefits of inhibiting IRAK-1 and Rac-1 interaction include, but are not limited to, blocking IRAK-1 and Rac-1 interaction to decrease the harmful generation of reactive oxygen species and decrease tissue damage during the process of inflammatory diseases such as atherosclerosis and lupus.

The assays disclosed herein to detect IRAK-1 and Rac-1 interaction can be used to screen for effective small compounds or other substances for the therapeutic purpose of treating inflammatory diseases. IRAK-1 can also serve as a diagnostic marker for the increased risk for atherosclerosis. Further, because it has now been discovered that certain IRAK-1 deletions have protective effects for diverse inflammatory diseases, detection of deletion mutants, for example by SNP analysis, can provide a diagnostic for septic shock, lupus, diabetes, and Alzheimer's disease. More specifically, healthy humans express the wild type version of IRAK-1 that runs at about 80 kDa on SDS-PAGE gels. Humans with high risks of developing diabetes and cardiovascular complications have the variant IRAK-1 that runs at about 100 dDa. The difference in size can thus be used as a determining factor in disease.

In summary, the present invention provides valuable insight in the molecular mechanism of a variety of diseases and disorders with a common regulatory point: IRAK-1. Biochemical assays for IRAK-1 functions are provided, which reveal that IRAK-1 activates Rac1 and NADPH oxidase; the LWPPPPSP motif of IRAK-1 is involved in its interaction with Rac1; IRAK-1 activates transcription factor C/EBPδ; IRAK-1 suppresses transcription factors RARα, LXRα, and NFAT. Cellular assays for IRAK-1 functions show that IRAK-1 is required for suppressing ABCA1 expression and cholesterol efflux in macrophages; IRAK-1 controls the balance of pro- and anti-inflammatory states of macrophages; and IRAK-1 is required for the differentiation of Th17 cells and the suppression of Treg cells.

### EXAMPLES

The invention will be further explained by the following Examples, which are intended to be purely exemplary of the invention, and should not be considered as limiting the invention in any way.

### Example 1

### Involvement of IRAK-1 with NFAT and Cardiovascular Disease

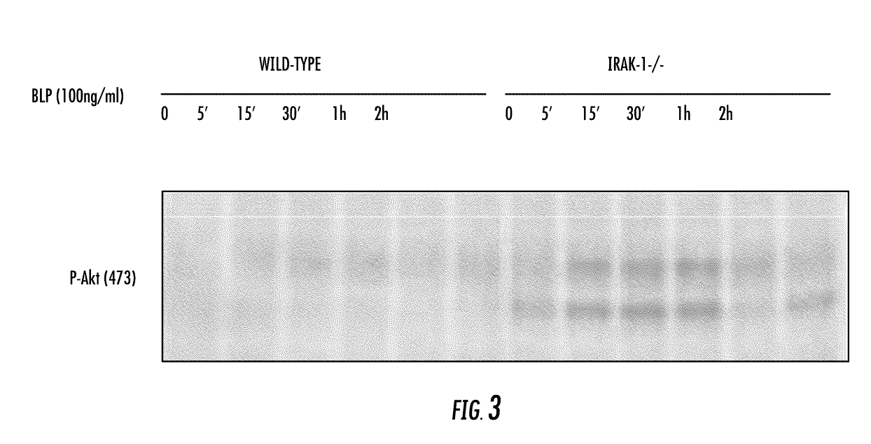

NFAT family transcription factors play critical roles in the pathogenesis of cardiovascular diseases, including hypertension and atherosclerosis. Because the inventor and his colleagues previously demonstrated that human IRAK-1 genetic variations are correlated with the risks of human cardiovascular diseases, contribution of IRAK-1 to the regulation of NFAT transcriptional activity was investigated. First, human Hela cells were trasfected with either empty vector plasmid or wild type IRAK-1 expression plasmid together with a NFAT responsive element-containing luciferase reporter plasmid. As can be seen from FIG. 2, introduction of a control empty plasmid and NFAT responsive element-containing luciferase plasmid led to the activation of the reporter luciferase activity, which is adjusted to 100% on the graph. As shown in FIG. 2, co-transfection of the wild type IRAK-1 expression plasmid with the NFAT-luciferase reporter plasmid led to decreased reporter luciferase activity (−80% reduction compared with the control transfection), indicating that IRAK-1 is suppressing NFAT activity. Subsequently, plasmids that express mutated IRAK-1 molecules (D340N, C203S, T113I, mutations which are present in human patients with cardiovascular diseases) have been generated. Mutated IRAK-1 molecules failed to suppress NFAT activity.

Further experiments were performed to ask whether the C-terminal domain of IRAK-1 is involved in facilitating its suppressive regulatory function toward NFAT activity. As shown in FIG. 2, co-transfection of IRAK-1-ΔC plasmid failed to suppress NFAT activity. IRAK-1b is an isoform of IRAK-1 that does not possess kinase activity. Experiments demonstrated that IRAK-1b cannot fulfill the inhibitory function on NFAT either. Statistical significance was calculated using a t-Test for two-samples assuming unequal variances and p-values are as indicated.

Suppression of NFAT activity has shown to be mediated by phosphorylation of NFAT. The present data therefore indicate that IRAK-1 is responsible for phosphorylating NFAT and subsequently inactivate NFAT activity. As can be seen from the data of FIG. 2, NFAT reporter activity is down-regulated by wild-type (wt) IRAK-1, but not by other constructs. In contrast, variant forms of IRAK-1 that are found in the human population (D340N, C203S, T113I) do not suppress NFAT activity. Furthermore, deletion of the C-terminus of IRAK-1 (IRAK-1 ΔC) also ablates its inhibitory effect on NFAT. Elevated NFAT activity is linked with cardiovascular disease. Thus, these results show that IRAK-1 and its derivatives can be used both in treatment of cardiovascular disease and in screening for drugs to treat cardiovascular disease.

### Example 2

### Involvement of IRAK-1 with Akt and Diabetes

Because Akt is a signaling molecule involved in regulating cellular metabolism, the activation status of Akt (as measured by the levels of its phosphorylation at Ser 473) in wild type and IRAK-1 deficient cells was investigated. Bone marrow derived macrophages (BMDM) were harvested from either wild type or IRAK-1 deficient mice. Equal amounts of BMDM (1×10⁶ cells) were treated with 100 ng/ml Pam₃CSK₄ (BLP) for a time course (0, 5 min, 15 min, 30 min, 1 hr, and 2 hr). Cell lysates were harvested from each time point and separated by electrophoresis. The intensities of phosphorylated Akt at Serine 473 position were monitored through Western Blot using anti-phosphorylated Akt Serine 473 antibody. The data indicate that IRAK-1 is attenuating agonist-induced Akt phosphorylation. This might be achieved through IRAK-1 phosphorylation and inactivation of Akt upstream molecules such as IRS-1.

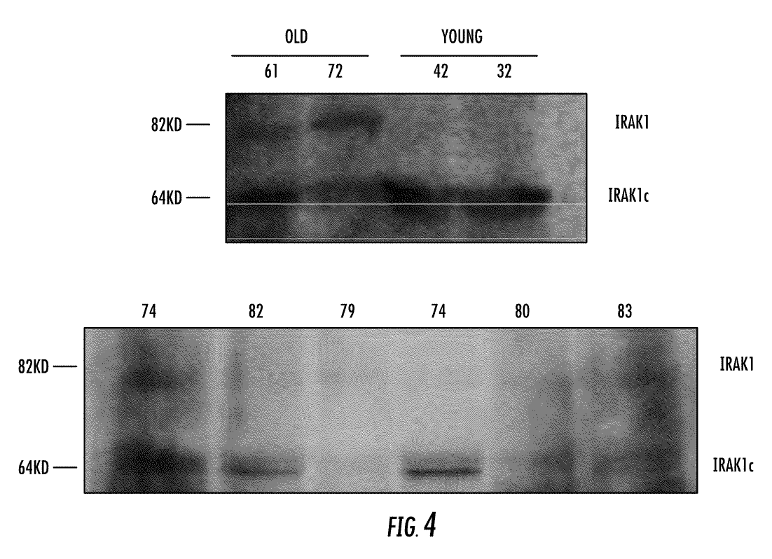

As can be seen from FIG. 3, IRAK-1 is involved in attenuating Akt activation. Decreased Akt activation is responsible for insulin resistance. Therefore, these results show that IRAK-1 and its derivatives can be used both in treatment of diabetes and insulin resistance and in screening for drugs to treat diabetes and insulin resistance.

### Example 3

### Involvement of IRAK-1 with Tau and Neurodegenerative Diseases

The expression pattern of full length IRAK-1 molecule and its inactive isoform IRAK-1c in human brain tissues were studied. Proteins were extracted from brain tissues from anonymous donors collected from Wake Forest University Medical Center. Harvested proteins from various donors with different ages were separated by electrophoresis. The levels of full-length IRAK-1 were visualized by Western Blot using anti-IRAK-1 antibody. As shown in FIG. 4, the full length IRAK-1 protein is not present in brain samples collected from young donors (age 42 and 32). In contrast, the full length IRAK-1 form is present in aged brain tissues (aged 61, 72, 74, 79, 80, 82, and 83).

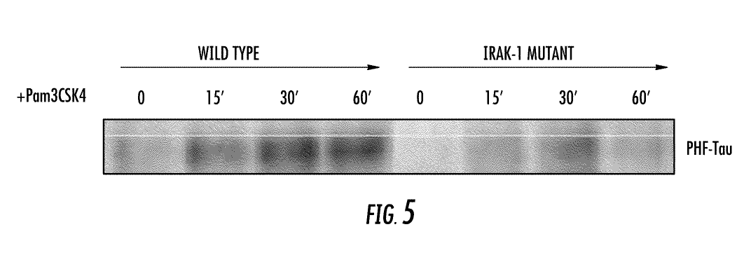

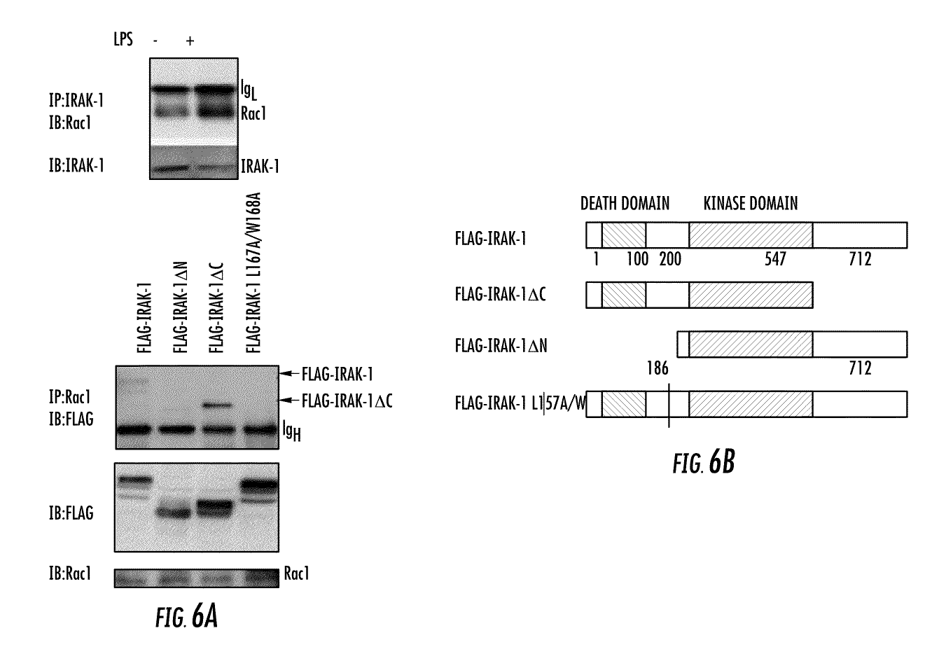

Because Tau phosphorylation is increased in aged human brains and contributes to the pathogenesis of various neurological diseases, such as Alzheimer's and Parkinson's diseases, studies were performed to ask whether the increased levels of full-length IRAK-1 can contribute to Tau phosphorylation. In order to answer this question, a transgenic mouse model known in the art was used. According to this study, either wild type or IRAK-1 deficient bone marrow derived macrophages were treated with BLP (Pam₃CSK₄) for various times, as indicated in FIGS. 4 and 5. The levels of phosphorylated Tau (PHF-Tau) were detected through Western Blot using anti-phosphorylated Tau antibody. As shown in FIG. 5, stimulation of wild type cells led to increased Tau phosphorylation. In contrast, Tau phosphorylation is ablated in IRAK-1 deficient cells. This study indicates that IRAK-1 is responsible for Tau phosphorylation.

The data in FIGS. 4 and 5 show that IRAK-1 is involved in phosphorylation of Tau by showing that cells without IRAK-1 have less phosphorylated Tau. Full-length Tau protein is found in adult, and in particular aged, human brains, whereas it is absent or present in small amounts in child, or young, brains. IRAK1c, the form found in young brains, is not active in phosphorylation of Tau, whereas IRAK-1 shows such an activity. Phosphorylated Tau protein shows reduced activity in maintenance of cytoskeletal structure, and is known to be involved in plaque formation and neurodegenerative disease. Thus, IRAK-1 is involved in neurodegenerative disease and it and its derivatives can be used in treatment of neurodegenerative disease and in screening for drugs to treat neurodegenerative disease.

### Example 4

### Interaction of IRAK-1 with Cellular Protein Rac1

One novel molecular target of IRAK-1 is the cellular protein Rac1. The data in FIG. 6 demonstrate that the LWPPPPSP motif of IRAK-1 is required for its interaction with Rac1.

More specifically, Panels A-C show data supporting this conclusion. Panel A: Wild-type murine BMDM cells were either untreated or treated with 100 ng/ml LPS for 5 min. Equal amounts of total cell lysates were harvested and used to perform immunoprecipitation analyses using an anti-IRAK-1 antibody. Co-immunoprecipitated protein complexes were resolved on a SDS-PAGE, and blotted with an anti-Rac1 antibody (top panel). A control anti-rabbit secondary antibody was used to perform a similar immunoprecipitation study, which gave no signal near the Rac1 region (data not shown). The levels of IRAK-1 in the cell lysates are shown in the bottom panel. Panel B is a diagrammatic illustration of various Flag tagged IRAK-1 full-length and deletion constructs used in the transfection studies. Panel C shows data from MAT4 cells that were transiently transfected with either pFlag-IRAK-1, pFlag-IRAK-1 ΔN, pFlag-IRAK-1 ΔC, or pFlag-IRAK-1(L167AW168A) mutant. Equal amount of lysates were harvested from the transfected cells and used to perform immunoprecipitation analyses using an anti-Rac1 antibody. Co-immunoprecipitated protein complexes were resolved on SDS-PAGE and blotted with an anti-Flag antibody (Top panel). A control anti-rabbit secondary antibody was used to perform a similar immunoprecipitation study, and did not give a non-specific signal near the region of interest (data not shown). The expression levels of Rac1 and various Flag-IRAK-1 mutants within the cell lysates are indicated in the bottom panels.

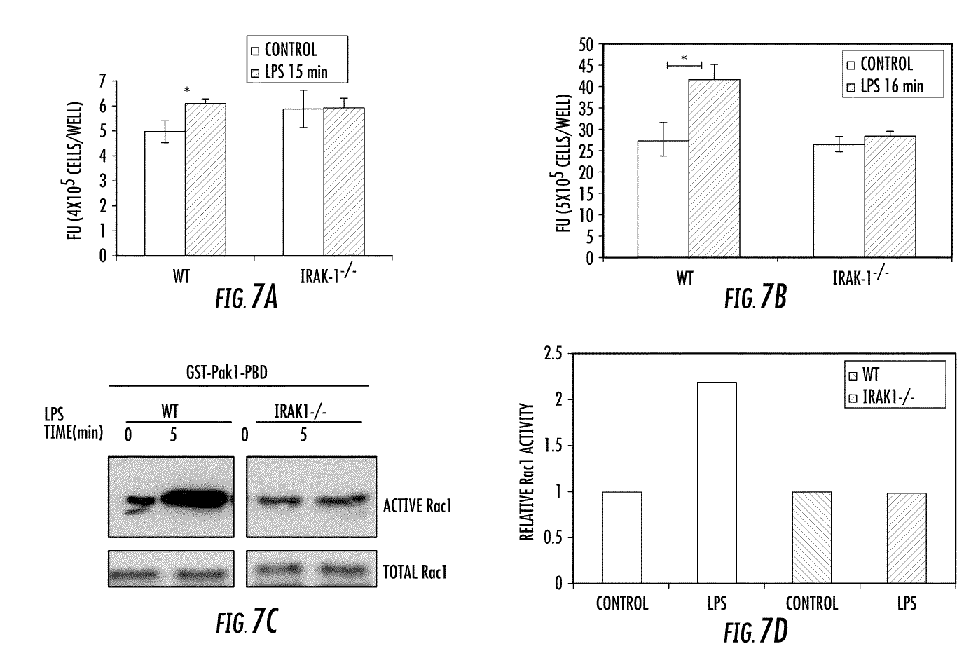

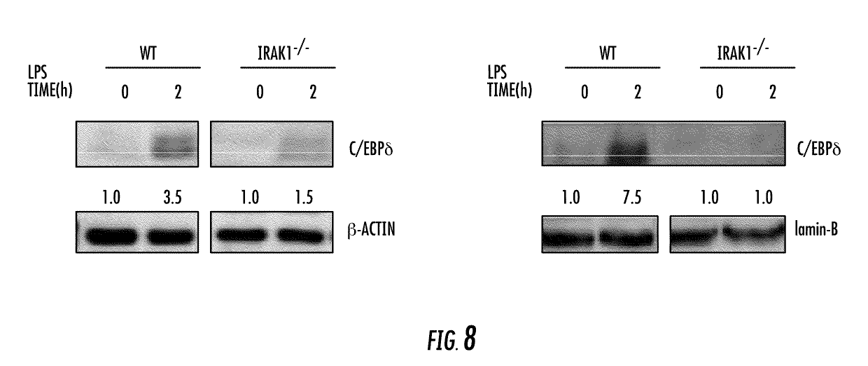

To determine if IRAK-1 is required for LPS-induced activation of Rac1, either wild type or IRAK-1 deficient macrophages were treated with LPS (100 ng/ml). FIG. 7 shows that LPS treatment induced significantly higher levels of reactive oxygen species in wild type macrophages, but not in IRAK-1 deficient macrophages with either 15 minutes of LPS treatment (Panel A) or 16 hours of treatment (Panel B). Additionally, Western blot analysis showed the LPS-induced activation of Rac1 by IRAK-1 after 5 minutes of LPS treatment (Panel C). No induction was seen in IRAK-1 deficient macrophages. Panel D demonstrates that relative Rac1 activity is significantly higher in LPS-induced wild type macrophages and not in IRAK-1 deficient macrophages. This data shows that IRAK-1 is required for LPS-induced activation of Rac1.

More specifically, the figure shows that IRAK-1 is involved in LPS-induced ROS formation. Panel A shows the effect of LPS on ROS production in WT and IRAK1^{-/-} BMDM cells. Intracellular ROS levels were measured by DCFDA staining using fluorescence microscope after LPS (100 ng/ml) stimulation in WT and IRAK1^{-/-} BMDM cells for 15 min. Panel B shows similar data obtained at 16 hours. *P<0.05.

The figure further shows that IRAK-1 is required for LPS-mediated activation of Rac1. Rac1 activity was determined using the PBD pull down assay following LPS stimulation (100 ng/ml) for 5 min in WT and IRAK1^{-/-} BMDM cells followed by immunoblotting with an anti-Rac1 antibody. The results are shown in Panel C, in which the bottom immunoblot panel shows total Rac1 expression in whole cell lysates, and Panel D shows the amount of activated Rac1 normalized to the amount of total Rac1 in whole cell lysates. The bar graphs are densitometric analyses of the active Rac1 specific bands from three independent experiments.

### Example 5

### IRAK-1 Activation of Transcription Factor C/EBPδ

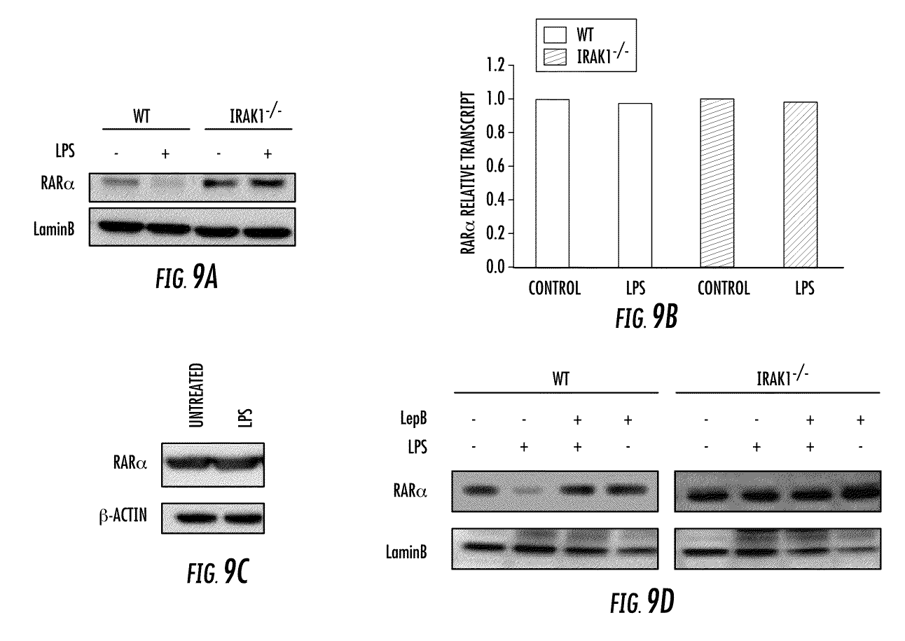

To determine if IRAK-1 is involved in LPS-induced C/EBPδ expression, either wild type or IRAK-1 deficient macrophages were treated with LPS for two hours. Western blot analysis in two different experiments demonstrated the induction of LPS after two hours with the wild type macrophages, but no induction with the IRAK-1 deficient macrophages (FIG. 8). Either β-actin or lamin-B proteins were used as controls. This data supports the involvement of IRAK-1 in LPS-induced C/EBPδ expression and nuclear translocation.

### Example 6

### IRAK-1 Suppression of Transcription Factor RARα

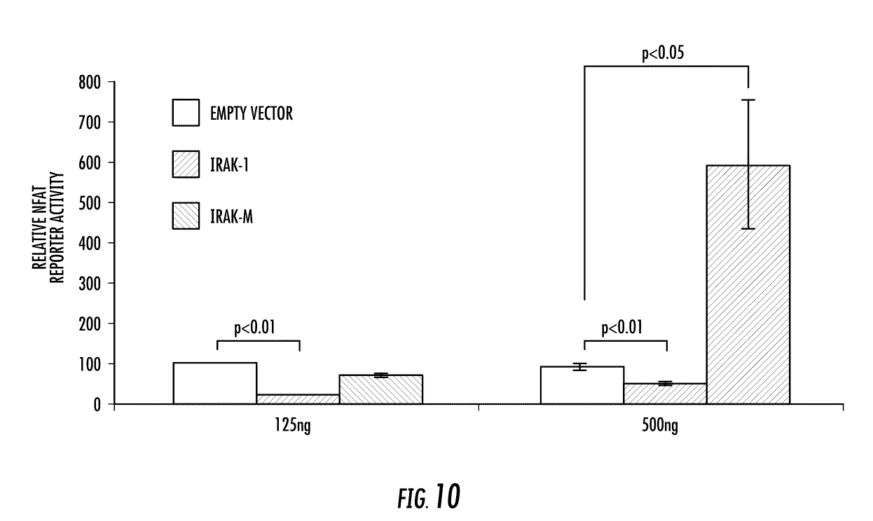

To determine if IRAK-1 is involved in the nuclear translocation of RARα, the levels of RARα were determined in wild type and IRAK-1 deficient macrophages in the presence and absence of LPS (FIG. 9). As seen by Western blot analysis, Panel A shows the suppression of RARα protein in LPS-induced wild type cells and no significant suppression in IRAK-1 deficient cells. Panel B demonstrates the RARα relative transcript levels in LPS-induced wild type or LPS-induced IRAK-1 deficient cells. In all samples, the relative transcription levels of RARα are approximately equal, suggesting that suppression by IRAK-1 of RARα does not occur at the transcriptional level. Panel C shows an additional experiment in which untreated and LPS-induced IRAK-1 deficient cells were tested for the presence of RARα protein by Western blot analysis. This experiment again demonstrates that RARα is not suppressed when IRAK-1 is not present. Panel D demonstrates the LPS-induced suppression of RARα by IRAK-1. When experiments are performed in a IRAK-1 deficient background, RARα is not suppressed (lanes 5-8). As expected, when a wild type background is used, IRAK-1 suppression can be seen (lanes 1 and 2). However, in the presence of LepB, RARα suppression by IRAK-1 is not seen (lanes 3 and 4).

More specifically, FIG. 9 shows the differential effect of LPS on nuclear RARα protein levels in WT and IRAK1^{-/-} BMDMs. Panel A shows the effect of LPS on nuclear RARα levels in BMDMs. WT and IRAK-1^{-/-}. BMDMs were treated with 100 ng/ml LPS for 2 h followed by nuclear protein extraction. The samples were analyzed by immunoblotting using the indicated antibodies. LaminB was used as the loading control. Panel B: the BMDMs derived from WT and IRAK-1^{-/-} mice were treated with LPS for 2 h followed by RNA extraction. The resulting cDNAs were used to detect RARα transcript levels by real time RT-PCR and standardized against GAPDH levels. Panel C: WT BMDMs were treated with 100 ng/ml LPS for 2 h followed by whole cell protein extraction. The samples were resolved by SDS-PAGE followed by immunoblotting with anti-RARα antibodies. Panel D: BMDM cells derived from WT mice were either untreated or treated with Leptomycin B (LepB) alone or in the presence of LPS. After 2 h incubation, nuclear lysates were prepared and subjected to SDS-PAGE followed by Western blot analysis with RARα specific antibodies. The blots were also probed with LaminB-specific antibodies as a loading control. ns=non-specific.

### Example 7

### IRAK-1 Suppression of Transcription Factor NFAT

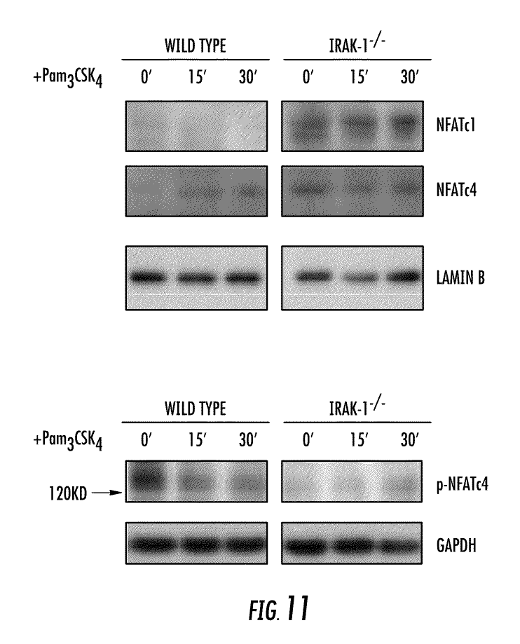

In order to determine the mechanism for IRAK-1 involvement, transcription factor reporter assays were performed. In this set of experiments, NFAT reporter activity was determined in the presence of IRAK-1 or IRAK-M, a homologue of IRAK-1 (FIG. 10). When 500 ng of vector was added, suppression of NFAT by IRAK-1 could be seen. However, IRAK-M, a homologue of IRAK-1, did not show suppression of NFAT.

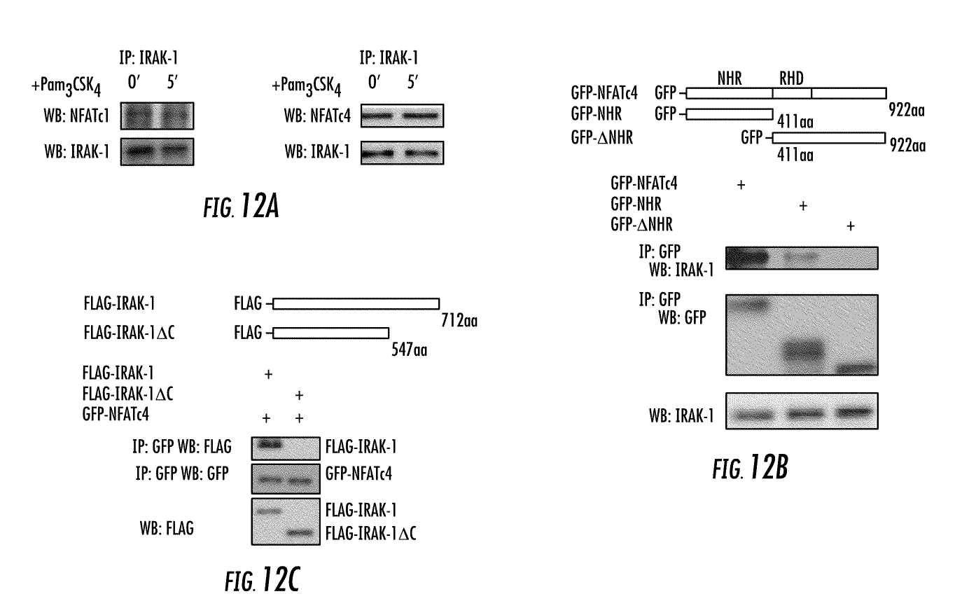

To help determine the molecular mechanism for IRAK-1 mediated regulation of NFAT, mutated versions of NFAT were tested (FIG. 11). NFATc1 and NFATc2 are two isoforms of NFAT family members that are present in multiple cells and tissues. Both NFATc1 and NFATc4 were suppressed in wild type cells comprising IRAK-1 (Panel A, lanes 1-3) and were not suppressed in IRAK-1 deficient cells (Panel A, lanes 4-6). However, when Western blot analysis was performed to detect phosphorylated NFATc4 (p-NFATc4), p-NFATc4 was found in the wild type cells (Panel B, lanes 1-3) whereas significantly less was found in the IRAK-1 deficient cells (Panel B, lanes 4-6). These set of experiments suggest that IRAK-1 is involved in the regulation of NFAT by its ability to phosphorylate NFAT.

To further delineate which part of IRAK-1 is involved in its interaction with NFAT, experiments were performed using full-length IRAK-1 and a C-terminally truncated form (IRAK-1ΔC—see FIG. 1). Specifically, Hela cells were transfected with IRAK-1 encoding plasmids that express either the full length wild type human IRAK-1 protein or a truncated version of IRAK-1 with deletion of the C terminal region (amino acids 548 to 712 are deleted). Equal amount of cell lysates were harvested from the transfected cells and used to perform co-immunoprecipitation assays with an anti-Flag antibody. Immunoprecipitated proteins were resolved by electrophoresis, and the co-immunoprecipitated proteins were subjected to Western blot analyses using an anti-NFAT antibody. As shown in the figure, only the full length version of the IRAK-1 molecule is capable of co-immunoprecipitating with NFAT.

### Example 8

### IRAK-1 Suppression of Transcription Factor PPARα

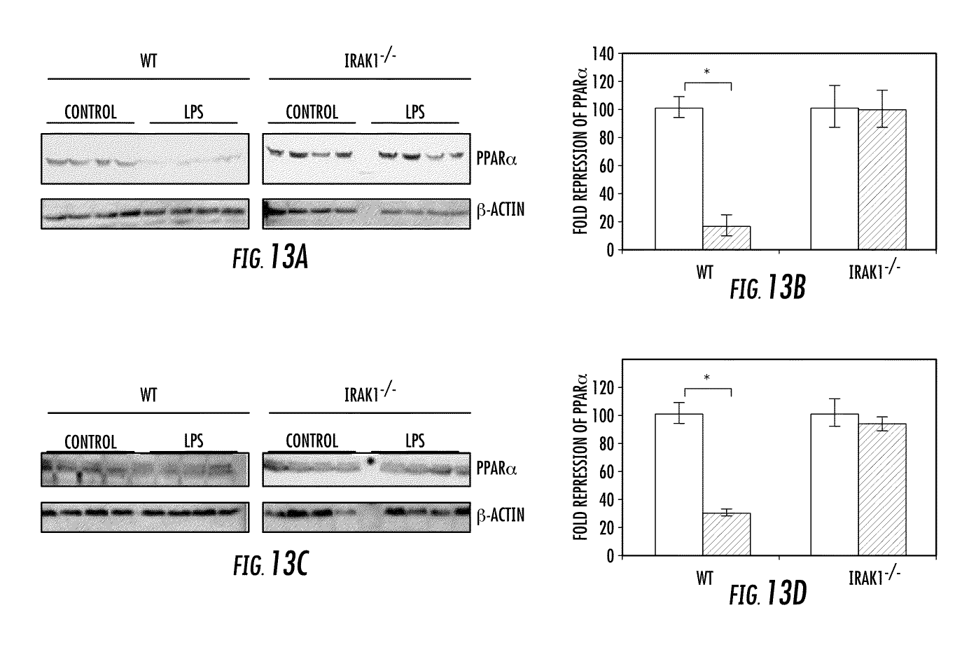

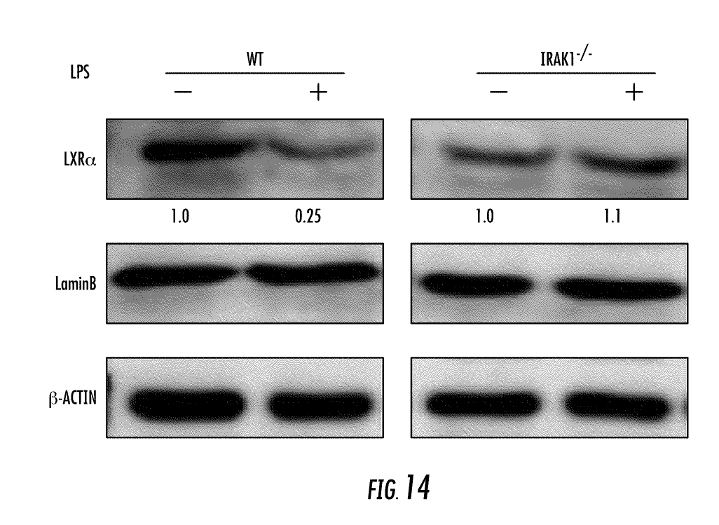

It was of interest to determine if the transcription factor PPARα is also regulated by IRAK-1. Experiments were performed toward that end, and the results are presented in FIG. 13. Specifically, Panel A shows Western blot analysis of samples from wild type and IRAK-1 deficient cells in the presence and absence of LPS. As can be seen, PPARα was detected in the wild type cells (Control; lanes 1-4) and not in the LPS-induced cells (lanes 5-8). However, PPARα was seen in both the Control and LPS samples in the IRAK-1 deficient cells (lanes 1-8). These experiments demonstrate that IRAK-1 suppresses the PPARα protein. A graphic portrayal of the same data showing the fold repression of PPARα can be seen in Panel B. A similar experiment again demonstrating IRAK-1 suppression of PPARα can be seen in Panels C and D.

### Example 9

### IRAK-1 Suppression of Transcription Factor LXRα

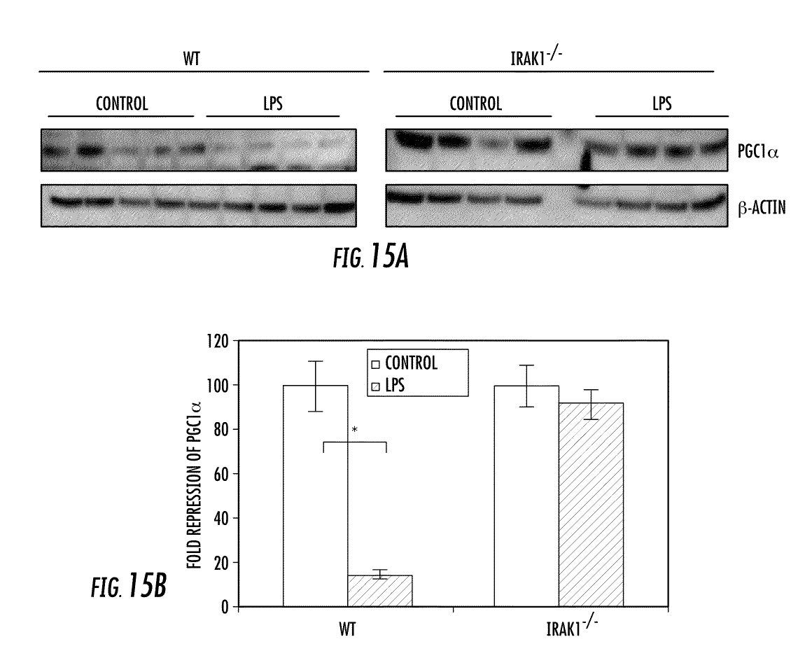

The transcriptional factor LXRα was also thought to be a potential target of IRAK-1 suppression. FIG. 14 illustrates Western blot analysis of the LXRα protein in the presence and absence of IRAK-1. Suppression of LXRα can be seen in the wild type cells (lanes 1 and 2). The amount of LXRα detected in the wild type cells without LPS induction was four times the amount found in the LPS-induced wild type cells, suggesting suppression of LXRα in the presence of LPS-induction. This difference was not seen in the IRAK-1 deficient cells, where the amount of LXRα was approximately the same in the presence or absence of LPS. Therefore, this data demonstrates that IRAK-1 suppresses the LXRα protein.

### Example 10

### IRAK-1 Suppression of Transcription Factor PGC1α

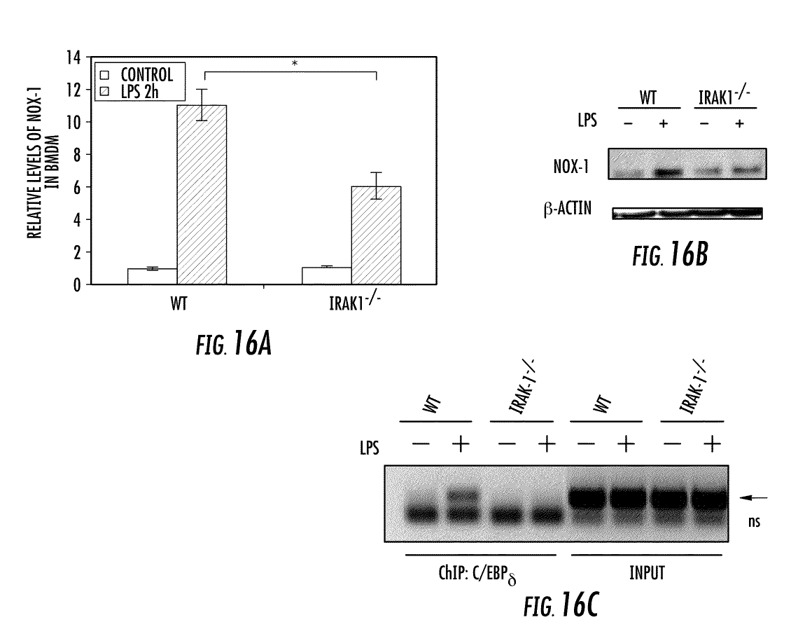

The ability of IRAK-1 to regulate the transcription factor PGC1α was determined in this set of experiments. PGC1α was detected by Western blot analysis in either wild type or IRAK-1 deficient cells in the presence or absence of LPS (FIG. 15). The samples without LPS from the wild type cells (Control; Panel A, lanes 1-5) showed significant amounts of PGC1α, whereas the LPS-induced samples from the wild type cells showed much less of the PGC1α protein (Panel A, lanes 6-9). The samples from the IRAK-1 deficient cells did not show the LPS-induced suppression (as seen in the second autoradiogram). A graphic illustration of these results is shown in Panel B. The fold repression of PGC1α can be seen in the wild type cells, but is insignificant in the IRAK-1 deficient cells, which do not contain the IRAK-1 protein. These results show that IRAK-1 suppresses the PGC1α transcriptional factor.

### Example 11

### IRAK-1 Induction of Inflammatory Mediators NOX-1 and MCP-1

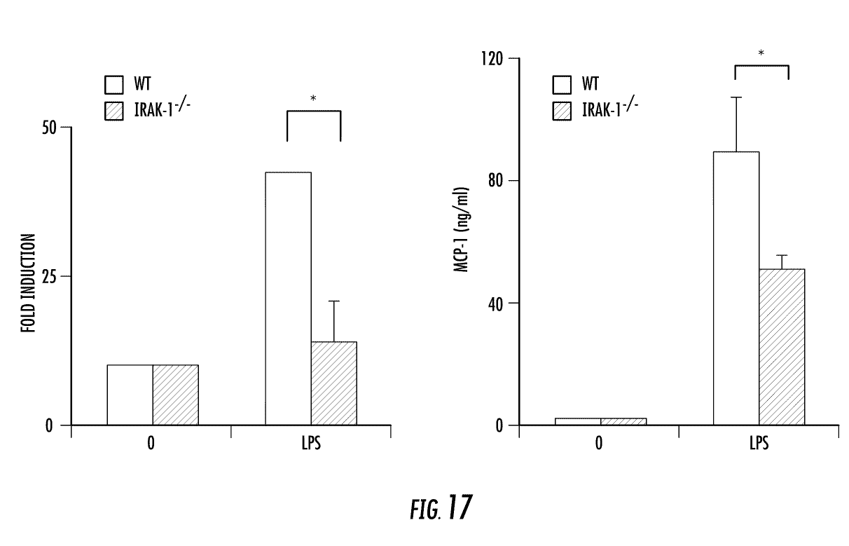

To determine if IRAK-1 was involved in the regulation of inflammatory mediator NOX-1, the levels of NOX-1 were determined in wild type and IRAK-1 deficient macrophages in the presence and absence of LPS (FIG. 16). Western blot analysis showed the LPS-induction of NOX-1 in wild type macrophage cells (Panel B, lanes 1-2). Induction was seen to a lesser extent in IRAK-1 deficient cells (Panel B, lanes 3-4). A graphic illustration of the amounts of NOX-1 detected in this experiment can be seen in Panel A. Levels of NOX-1 found in the LPS-induced wild type cells were significantly higher than the levels found in the IRAK-1 deficient background, showing that IRAK-1 is involved in the induction of NOX-1.

More specifically, FIG. 16 shows that IRAK-1 contributes to LPS-induced expression of NOX-1. Panel A: Effect of LPS on NOX-1 expression in WT and IRAK1^{-/-} BMDM cells. The cells were stimulated with LPS (100 ng/ml) for 2 h. After stimulation, total RNA was prepared using Trizol reagent followed by reverse transcription and the mRNA levels of NOX-1 were analyzed using real-time RT-PCR. Each data point represents the mean+/−standard deviation of at least three independent experiments. *P<0.05, compared with control. Panel B: The protein levels of NOX-1 were analyzed after LPS stimulation in WT and IRAK1^{-/-} BMDM cells by Western blot using an anti-NOX-1 antibody. The same blots were probed with β-actin as the loading control. Panel C: Chromatin immunoprecipitation analyses were performed to determine LPS-inducible recruitment of C/EBPδ to the promoter region of NOX-1. In contrast, LPS cannot induce C/EBPδ binding to the promoter region of NOX-1 in IRAK-1 deficient macrophages.

### Example 12

### IRAK-1 Induces the Expression of MCP-1 Via CEBPδ

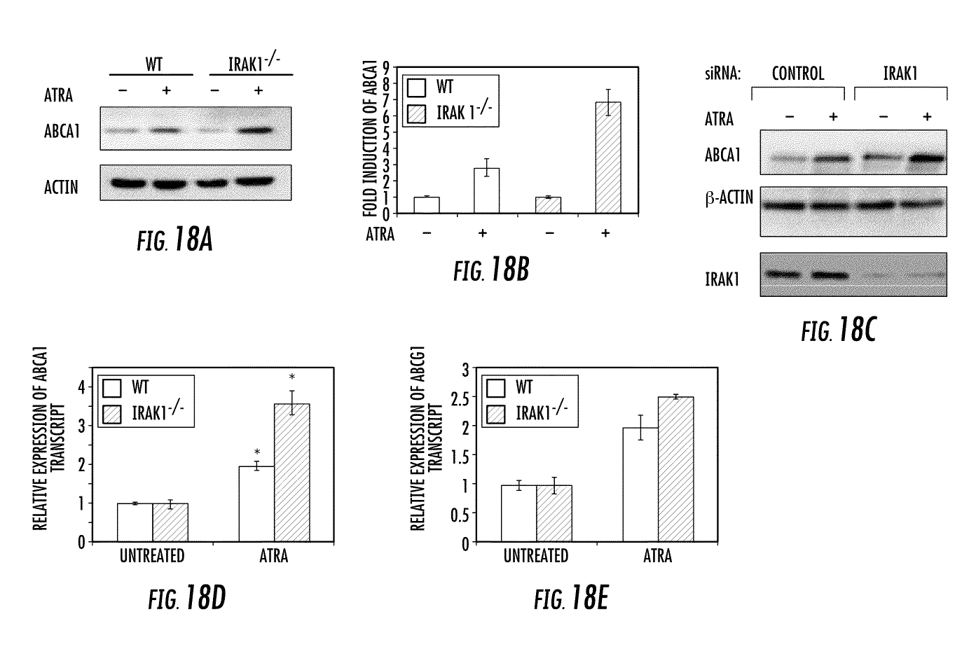

To investigate the possible role of IRAK-1 in control of MCP-1 expression, experiments were performed to assess the level of MCP-1 produced via LPS stimulation. It was postulated that IRAK-1 regulates the expression of MCP-1 through interaction of IRAK-1 with C/EBPδ. To assess this possible interaction, MCP-1 mRNA levels were determined in both wild-type cells and IRAK^{-/-} cells in the presence and absence of LPS. The results are depicted graphically in FIG. 17.

As shown in the figure, the levels of MCP-1 message (left panel) and protein (right panel) in wild type and IRAK-1 deficient macrophages following LPS stimulation was measured by real-time PCR analyses (left panel) and ELISA (right panel). LPS stimulation of wild-type cells resulted in a significant increase in induction of MCP-1 transcription and protein expression. The data fully support a role for IRAK-1 in control of expression of MCP-1 transcription through control of C/EBPδ.

### Example 13

### IRAK-1 Suppression of Expression of ABCA1 in Macrophages

To further characterize the role of IRAK-1 in immunity and inflammation, the possibility of a role for IRAK-1 in foam cell formation was investigated. The results are presented in FIG. 18, which shows that loss of IRAK-1 increases ABCA1 mRNA and protein levels in response to ATRA.

Panel A shows that there are increased levels of ABCA1 protein in BMDMs lacking IRAK1 in response to ATRA. WT and IRAK1^{-/-} BMDM cells were either untreated or treated with ATRA (50 nM) followed by Western blot analysis of cell extracts using ABCA1 specific antibodies. Antibodies against β-actin were used as the internal loading control. Panel B: the band intensities were quantitated using the Fujifilm Multi Gauge software and the fold induction is depicted after normalization against β-actin levels. Panel C: IRAK-1 expression was knocked-down using IRAK-1 specific siRNA (IRAK1). Wild type macrophages treated with either control siRNA or IRAK-1 specific siRNA were stimulated with 50 nM ATRA for 6 hrs. The levels of ABCA1 protein were determined by Western blot using anti-ABCA1 antibody. The levels of beta-actin were probed and served as equal loading controls. The levels of IRAK-1 were probed to make efficient knock-down with the IRAK-1 specific siRNA treatment. Panels D and E: higher induction of ABCA1 mRNA by ATRA in IRAK1^{-/-} BMDMs. The cells were 22 either untreated or treated with 50 nM ATRA for 6 h and the expression of ABCA1 and ABCG1 transcripts were measured by real time RT-PCR assays and standardized against GAPDH levels. Each experiment was performed in triplicate. *P<0.05 As can be seen from the data, IRAK-1 suppresses the expression of ABCA1 in macrophages, and thus plays an important role in regulation of foam cell formation.

### Example 14

### IRAK-1 is a Regulator of T Cell Development

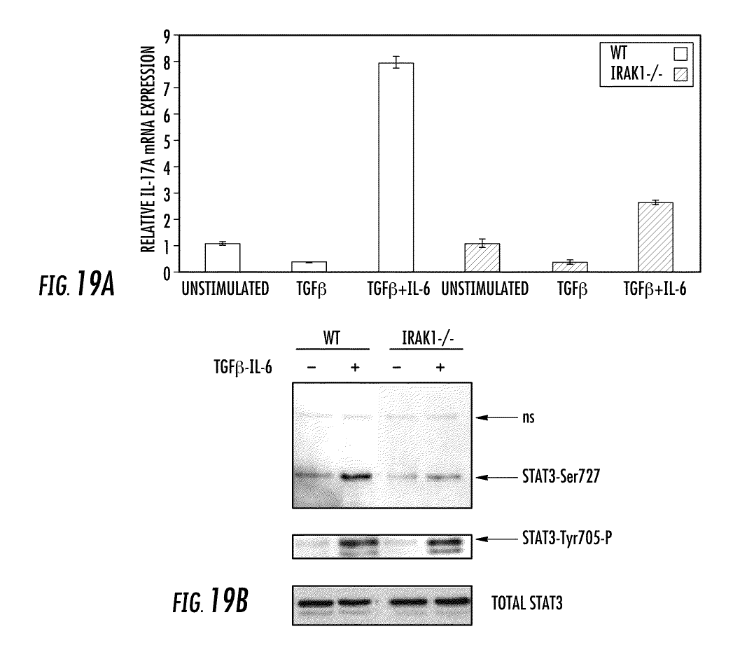

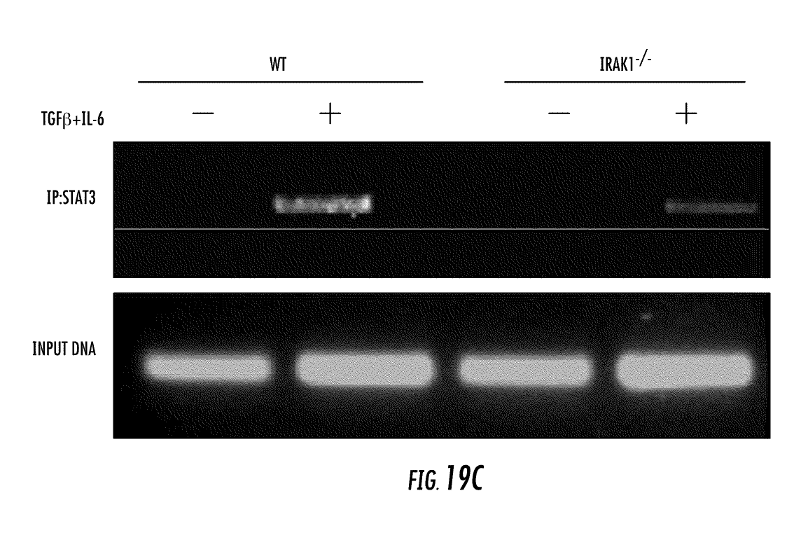

T cells play an important role in the development of inflammation. To assess the role of IRAK-1 in T cell development, IRAK-1 null mutant cells were assayed for IL-17 production upon stimulation with various agents. The results are depicted in FIG. 19. In summary, the results show that IRAK-1 is involved in IL-17 production by T cells.

More specifically, FIG. 19 provides data showing that there is decreased induction of IL-17A and RORγt in IRAK-1^{-/-} T cells in response to IL6 and TGFβ1. Panel A: naive CD4 T cells from wildtype (WT) and IRAK-1-deficient mice were stimulated with anti-CD3 and anti-CD28 for 3 days with or without TGFβ1 together with IL-6 as indicated. Expression of IL-17A mRNA was analyzed by real-time RT-PCR. Results represent the mean±SD of three independent samples. Results are expressed as mean±SD from three independent experiments. *, p<0.05; **, p<0.01. Panels B and C further show the differential effect of IL-6 on STAT3 phosphorylation in CD4 T cells isolated from wild-type (WT) and IRAK-1-deficient mice. Panel B: naive CD4 T cells were stimulated with TCR agonists in the presence or absence of TGFβ and IL-6. Cell lysates were harvested for the determination of STAT3 phosphorylation status (Ser727 and Tyr705) by immunoblotting. The same blots were probed with STAT3-specific Abs to compare the total levels of STAT3 in wild-type and IRAK-1-deficient T cells. ns=nonspecific bands. Panel C: chromatin immunoprecipitation (IP) assays were performed in unstimulated cells or cells stimulated with TGFβ and IL-6 plus anti-CD3 and anti-CD28 using STAT3-specific Abs. The input DNA was used as the loading control. Data are representative of three independent experiments.

### Example 15

### IRAK-1 Involvement in IL-17 Expression

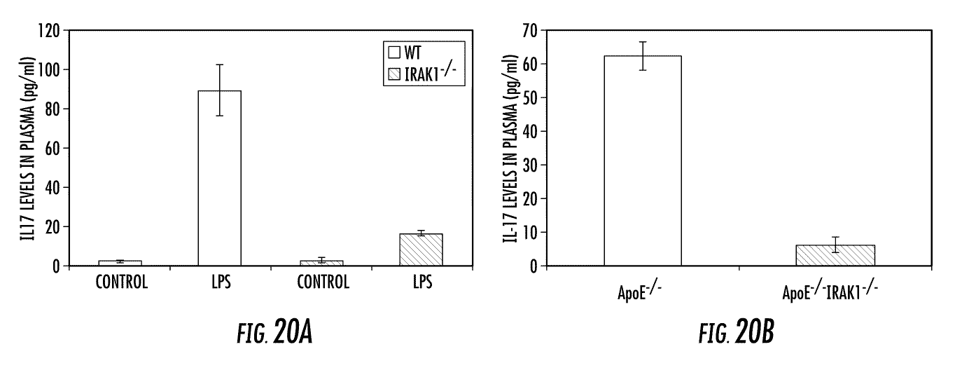

The previous Examples provide ample support for the involvement of IRAK-1 in the inflammatory, and thus immune, response. To further characterize the involvement of IRAK-1 in these processes, experiments were performed to determine the effect of IRAK on differentiation and function of T cells. To this end, the effect of IRAK-1 on production of IL-17 was determined. The results are presented in FIG. 20.

More specifically, FIG. 20 shows that there is decreased IL-17 expression and reduced inflammatory responses in IRAK-1-deficient mice. Panel A: wild-type (WT) and IRAK-1^{-/-} mice (n=4 per group) were injected with 500 μg LPS or PBS (Control) i.p. followed by isolation of plasma 6 h post injection. IL-17 levels were assayed using a Bio-Rad multiplex bead-based immunoassay kit. *, p<0.05. Panel B: ApoE^{-/-} and ApoE^{-/-}/IRAK-1^{-/-} mice were fed with a high-fat diet for 3 mo and the plasma levels of IL-17 were measured using the Bio-Rad multiplex bead-based assay kit. At least four mice were analyzed in each group. Results are expressed as mean±SD. *, p<0.01.

### Example 16

### IRAK-1 Deficient T Cells have Elevated Induction of Foxp3

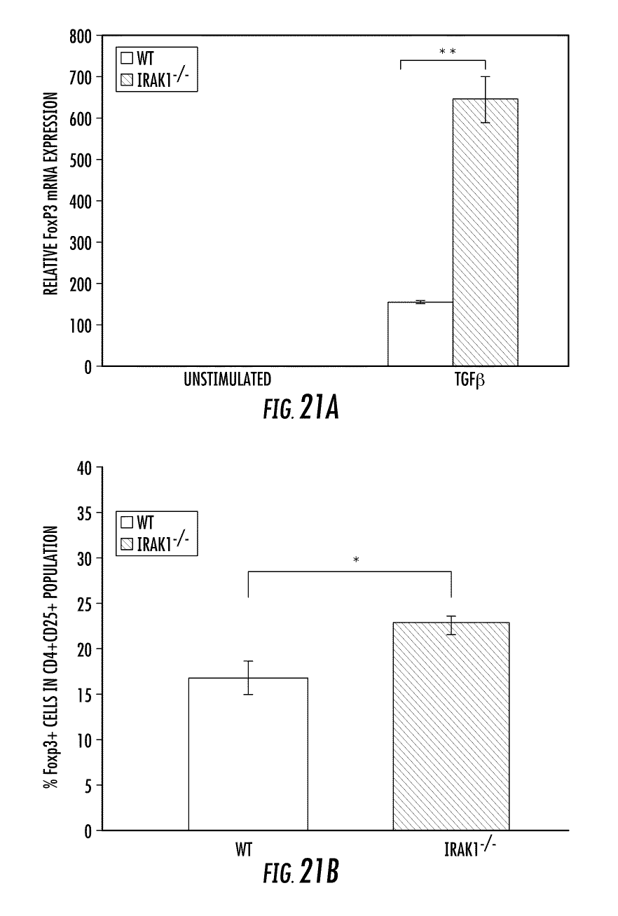

To characterize the role of IRAK-1 in T cell differentiation, IRAK-1 deficient cells were stimulated with TGFβ, and the expression of Foxp3 mRNA assayed. Foxp3 is known to be an indicator of differentiation of T cells to T regulator cells and not to T helper cells. The results of the experiments are shown in FIG. 21.

The figure shows that IRAK-1 is involved in suppressing the differentiation of T regulatory cells. Panel A: CD4 T cells from either wild type (WT) or IRAK-1 deficient mice were treated with T regulatory cell favoring conditions (anti-TGFβ, anti-CD3, anti-CD28) for two days. The levels of Foxp3 were measured using real-time PCR analyses. IRAK-1 deficient cells express significantly more Foxp3 (a marker for T regulatory cells). Panel B: Total splenocytes from WT and IRAK-1 deficient mice were labeled with antibodies against CD4, CD25, and Foxp3, and the cells were analyzed using flow cytometry. The Panel shows that IRAK-1 deficient mice have significantly higher levels of Foxp3 positive T regulatory cells.

### Example 17

### Deletion of IRAK-1 Protects Mice from Developing Insulin Resistance and Atherosclerosis

The role of IRAK-1 in inflammation is clear from the above data. The molecular mechanisms of IRAK-1 control of inflammation and the immune response has been detailed. However, to further investigate its role in inflammatory diseases, experiments were performed to determine its role in insulin resistance and atherosclerosis, both of which involve an inflammatory response.

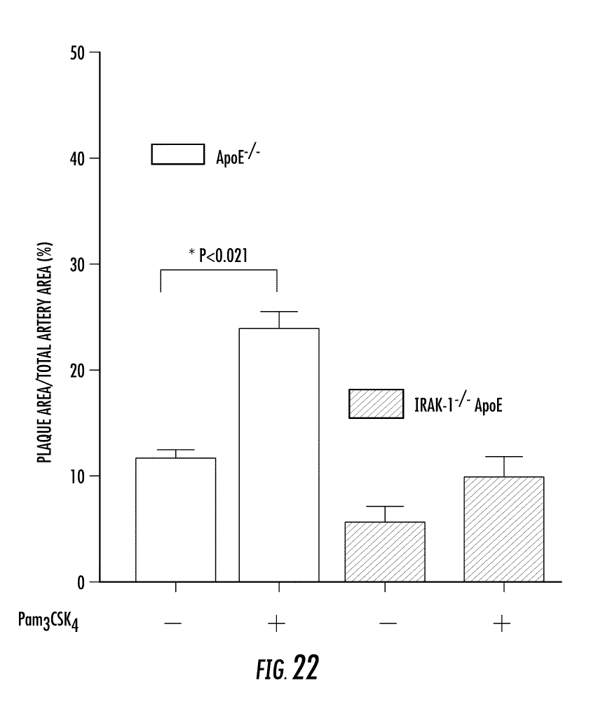

FIG. 22 provides data indicating that IRAK-1 deficiency alleviates the progression of atherosclerosis induced by TLR agonists. ApoE deficient and ApoE/IRAK-1 double deficient mice were fed with high-fat diet (Harlan, TD88137) with or without weekly injection of TLR2 agonist Pam3CSK4 for three month. At the end of the feeding regimen, aorta were dissected and the areas of lipid plaques were stained, visualized, and measured with Sudan IV. As can be seen from the Figure, there is a significant difference in the plaque area in IRAK^{-/-} aortic tissue. IRAK-1 is thus involved in progression of atherosclerosis.

### Example 18

### IRAK-1 is Activated in Human Leukocytes With Atherosclerosis

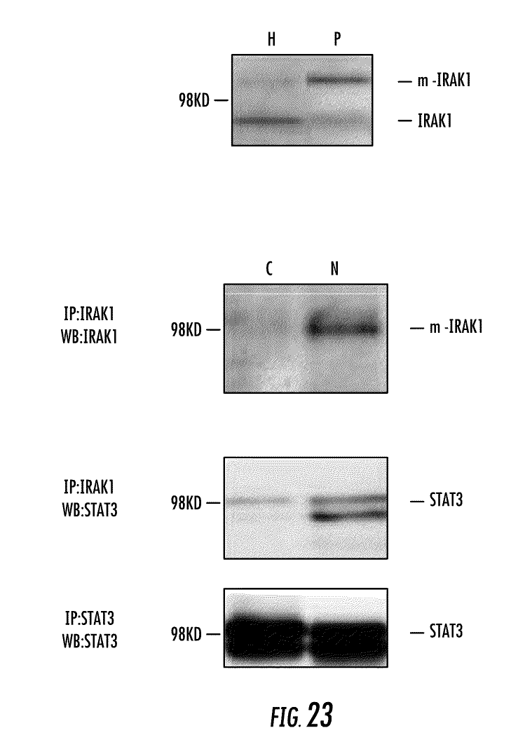

To further determine the role of IRAK-1 in atherosclerosis, the level of IRAK-1 in human leukocytes from atherosclerosis tissue was determined. The results are presented in FIG. 23.

The top panel of FIG. 23 shows that the IRAK-1 molecule is constitutively modified and activated in blood cells collected from human atherosclerotic patients. Human peripheral blood cells were harvested from either healthy donors (H) or atherosclerotic patients who had undergone angioplasty. Total cell lysates were harvested and analyzed by Western blot for the levels of either resting and unmodified IRAK-1 (IRAK-1) or the activated and modified IRAK-1 (m-IRAK-1). As can be seen, healthy donors showed expression of unmodified IRAK-1, whereas atherosclerotic patients showed expression of activated and modified IRAK-1. Further, the bottom panel shows that there is increased interaction of IRAK-1 with STAT3 in blood cells collected from atherosclerotic patients. IRAK-1 proteins were immunoprecipitated from harvested cell lysates and analyzed for the presence of STAT3 by Western blot.

### Example 19

### Genetic Markers for Cardiovascular Disease

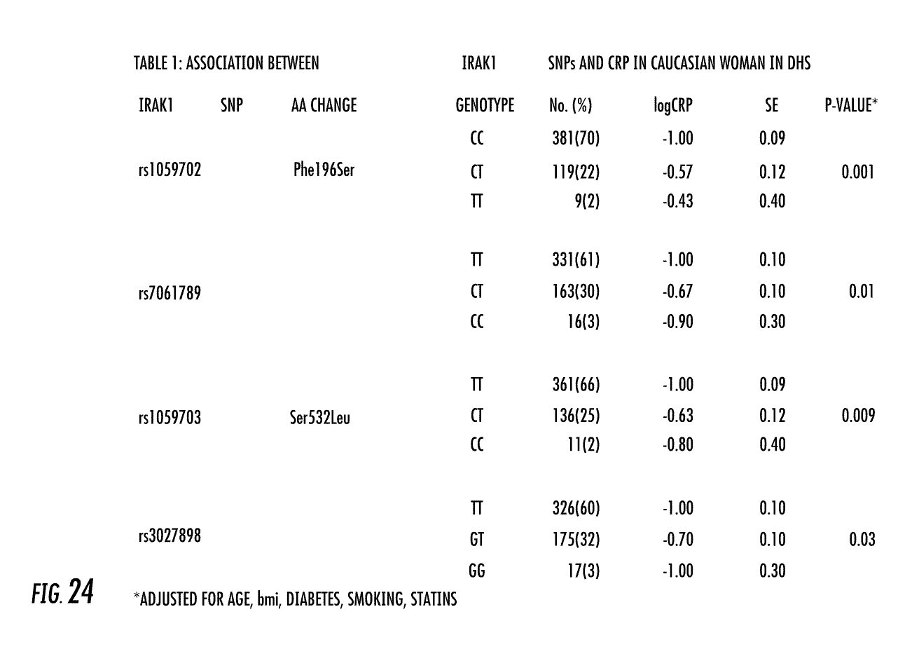

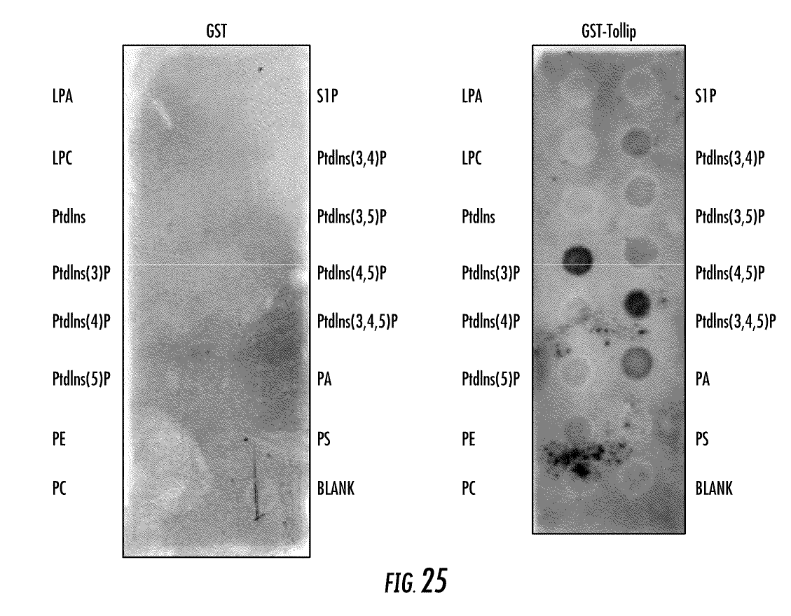

IRAK-1 plays a role in development of cardiovascular diseases. It has been determined that mutant forms of IRAK-1 exist in the human population. Several mutants were analyzed, and point mutations (single nucleotide polymorphisms; SNPs) were detected. FIG. 24 provides a summary of the analyzed SNPs and shows that two particular point mutations are correlated with cardiovascular disease. As can be seen in the figure, an F196S mutation and an S532L mutation correlate with atherosclerosis in the population studied. Accordingly, IRAK-1, and in particular the sequence of the IRAK-1 gene and protein, can be used as markers for atherosclerosis. Screening assays to detect the nucleic acid point mutations and the amino acid substitutions thus can be used to determine atherosclerosis in human populations.

### Example 20

### Involvement of Tollip

Tollip is an novel adaptor molecule capable of interacting with IRAK-1. Tollip deficient cells share similar phenotypes as IRAK-1 deficient cells. We show in this Example that Tollip can specifically interact with PI3P, which can serve as a tool to intervene the function of Tollip and IRAK-1.

### Example 21

### Integrated Role of IRAK-1 in T Cell Maturation, Foam Cell Formation, and the Inflammatory Process in General

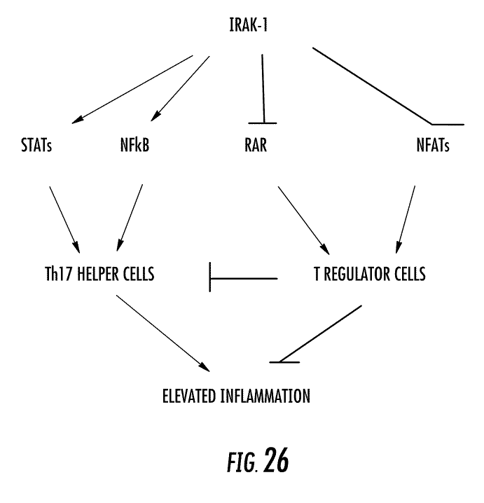

The data presented herein provides a conclusive role for IRAK-1 in development of T cells, and thus a role for IRAK-1 in the inflammatory process. FIG. 26 provides a summary of the data presented herein. As seen in the Figure, IRAK-1 has a positive effect on the activity of STATs and NFκB. These molecules in turn activate gene transcription for pathways that cause T cell differentiation and proliferation into T helper cells. As is well recognized in the art, T helper cells are important in pro-inflammatory processes. IRAK-1 is thus a key signalling and control point for T helper cell promotion and activity.

In contrast, as shown in the figure, IRAK-1 plays an inhibitory role in development of T regulatory cells. Specifically, IRAK-1 inhibits the activity of RAR and NFATs. These molecules are known in the art as important regulators of development of T cells into T regulator cells, which are involved in suppression of inflammation. The combined effects of IRAK-1 activity are thus to promote inflammation through T helper cells while at the same time inhibiting inflammation suppression through T regulator cells. IRAK-1 is thus a key control and signalling point in the development of inflammation. It thus is a key control point in progression of diseases and disorders involving inflammation. Use of IRAK-1 in assays for substances that block its interaction with STATs, NFκB, RAR, and NFATs can thus identify substances that can be used to control the inflammatory process and mitigate the deleterious effects of inflammation in numerous diseases and disorders. It is recognized that such molecules might not necessarily be fully therapeutic for treatment of inflammation and associated diseases and disorders; however, such molecules can provide, if not complete therapeutic results, at least some therapeutic effect.

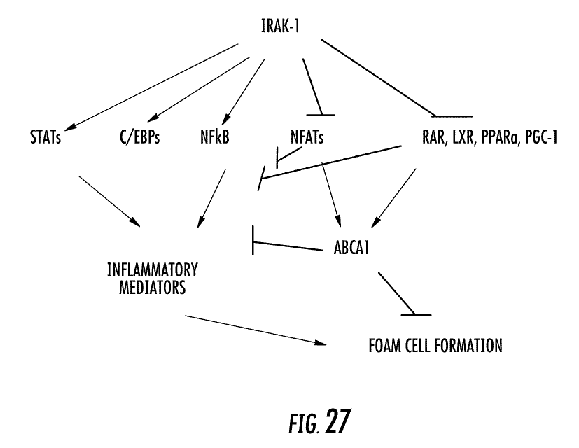

In addition to its role in inflammation via T cells, the data provided herein further shows that IRAK-1 is a key control point for macrophage involvement in inflammation. FIG. 27 provides a summary of the data developed herein, and shows clearly the role of IRAK-1 in controlling foam cell formation from macrophages. It is well recognized in the art that foam cells are an important aspect of inflammation. Looking at the figure in detail, it can be seen that IRAK-1 plays an activating role in the activity of STATs, C/EBPs, and NFκB. These molecules are known in the art to positively regulate the production of inflammatory mediators. These inflammatory mediators affect macrophage activity and morphology, causing production of foam cells from the macrophages. IRAK-1 thus directly activates biochemical processes that promote inflammation and the negative effects of inflammation on macrophages and the healing process.

At the same time, IRAK-1 has been shown herein to be a negative regulator of NFATs, RAR, LXR, PPARα, and PGC-1. These molecules are known in the art as negative regulators of the production of inflammatory mediators and/or as activators of ABCA1, which is an inhibitor of inflammatory mediators and foam cell production. IRAK-1 thus plays a dual role in suppression of foam cell production.

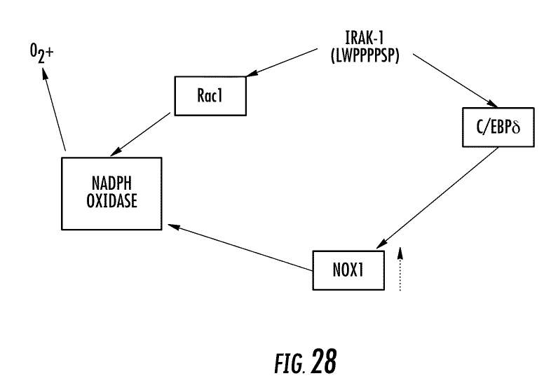

FIG. 28 provides a summary of the role of IRAK-1 in production of reactive oxygen species, which are known as key mediators in inflammatory processes. As shown in the figure, IRAK-1, through its characterized binding region, interacts with C/EBPδ to cause increased production of NOX1. The increase in NOX1 affects NADPH oxidase to cause an increase in the production of reactive oxygen species. At the same time, IRAK-1 interacts with Rac1 to cause yet a further increase in the activity of NADPH oxidase and yet a further increase in reactive oxygen species production.

It will be apparent to those skilled in the art that various modifications and variations can be made in the practice of the present invention without departing from the scope or spirit of the invention. Other embodiments of the invention will be apparent to those skilled in the art from consideration of the specification and practice of the invention. It is intended that the specification and examples be considered as exemplary only.

### SEQUENCE LISTING

SEQ ID NO:1 (human IRAK-1):

1 maggpgpgep aapgaqhfly evppwvmcrf ykvmdalepa dwcqfaaliv rdqtelrlce

61 rsgqrtasvl wpwinrnary adlvhilthl qllrardiit awhppaplps pgttaprpss

121 ipapaeaeaw sprklpssas tflspafpgs qthsgpelgl vpspaslwpp ppspapsstk

181 pgpessysll qgarpfpfcw plceisrgth nfseelkige ggfgcvyrav mrntvyavkr

241 lkenadlewt avkqsfltev eqlsrfrhpn ivdfagycaq ngfyclvygf lpngsledrl

301 hcqtqacppl swpqrldill gtaraiqflh qdspslihgd ikssnvllde rltpklgdfg

361 larfsrfags spsqssmvar tqtvrgtlay lpeeyiktgr lavdtdtfsf gvvvletlag

421 qravkthgar tkylkdlvee eaeeagvalr stqstlqagl aadawaapia mqiykkhldp

481 rpgpcppelg lglgqlaccc lhrrakrrpp mtqvyerlek lqavvagvpg hseaascipp

541 spqensyvss tgrahsgaap wqplaapsga saqaaeqlqr gpnqpvesde slgglsaalr

601 swhltpscpl dpaplreagc pqgdtagess wgsgpgsrpt aveglalgss asssseppqi

661 iinparqkmv qklalyedga ldslqllsss slpglgleqd rqgpeesdef qs

### SEQ ID NO:2:

### LWPPPPSP

### SEQ ID NO:3:

### SSSS

### SEQ ID NO:4

### Triacylated Cys-Ser-Lys-Lys-Lys-Lys (CSKKKK)

### SEQ ID NO:5

### PSPASLWPPPPSPAP

### SEQ ID NO:6

LWPPPP

1. A method of identifying a substance affecting inflammation, said method comprising:
combining the substance, IRAK-1 or a portion thereof having substrate-binding activity, and a substrate for IRAK-1, and
determining if the IRAK-1 and substrate can bind in an enzyme-substrate complex.

2. The method of claim 1, wherein the substrate is a STAT, NFκB, RAR, NFAT, C/EBPδ, LXR, PPARα, PGC1, or Rac1.

3. The method of claim 1, wherein the step of determining comprises detecting a complex of IRAK-1 and the substrate.

4. The method of claim 1, wherein the step of determining comprises detecting a change in the phosphorylation state of the substrate.

5. The method of claim 1, wherein the step of determining comprises detection of a gene expression product under the transcriptional control of the substrate.

6. The method of claim 5, wherein the gene expression product is a polypeptide.

7. The method of claim 6, wherein the gene expression product is an inflammatory mediator.

8. The method of claim 7, wherein the gene expression product is IL-17.

9. The method of claim 5, wherein the gene expression product is an mRNA transcript.

10. The method of claim 1, wherein the step of determining comprises detection of the differentiation state of a cell.

11. The method of claim 10, wherein the step of determining comprises determining if a T cell is a T helper cell or a T regulator cell.

12. The method of claim 10, wherein the step of determining comprises determining if a macrophage is destined to become a foam cell or physiological macrophage.

13. The method of claim 1, wherein the step of determining comprises detecting suppression of fatty acid oxidation.

14. The method of claim 13, wherein the step of determining occurs using a liver or kidney cell.

15. The method of claim 1, wherein the step of determining comprises detecting the production of reactive oxygen species.

16. A method of treating a subject suffering from a disease or disorder involving inflammation as a result of the activity of IRAK-1, said method comprising:
exposing at least one cell involved in the inflammation to a substance that alters the interaction of IRAK-1 with one of its substrates, in an amount effective to alter the interaction of IRAK-1 and the substrate(s),
wherein the substance reduces or eliminates aspects of inflammation.

17. The method of claim 16, wherein the method reduces the production of T helper 17 cells.

18. The method of claim 16, wherein the method increases the production of T regulator cells.

19. The method of claim 16, comprising:
removing from the subject one or more cells or cell types involved in the inflammation,
exposing the cell(s) to the substance to alter the cell(s); and
reintroducing the altered cell(s) into the subject.

20. A method of regulating the differentiation state of a cell, said method comprising:
exposing the cell to a substance that alters the interaction of IRAK-1 with one or more of its substrates under conditions whereby the substance can cause an alteration in the interaction of IRAK-1 with its substrate(s) and/or its downstream target functions, as well as the cellular responses to other inflammatory agents.

21. The method of claim 20, which is a method of regulating T cell differentiation.

22. The method of claim 20, which is a method of regulating foam cell formation.

23. The method of claim 20, which is a method of regulating fatty acid oxidation in metabolic cells.

24. A method of regulating the differentiation state of a cell, said method comprising:
exposing the immune cell to a substance that alters the interaction of IRAK-1 with one or more of its substrates under conditions whereby the substance can cause an alteration in the function of IRAK-1 in terms of its downstream target functions, as well as the cellular responses to other inflammatory agents.

25. The method of claim 24, which is a method of cellular responses to TLR agonists.

26. The method of claim 25, wherein the TLR agonist is LPS.

27. The method of claim 24, which is a method of cellular responses to nuclear receptor agonists.

28. The method of claim 27, wherein the agonist is ATRA.

29. A method of identifying a substance affecting inflammation, said method comprising:
combining the substance with lipopolysaccharide (LPS), TLR agonists, and/or nuclear receptor agonists in culture medium of wild type and IRAK-1 deficient cells, and
determining if the substance can affect cellular responses to LPS, TLR agonists, and/or nuclear receptor agonists in an IRAK-1 dependent fashion.

30. The method of claim 29, wherein the agonist is all trans retinoic acid (ATRA).

31. The method of claim 29, wherein the cellular response is the expression of MCP-1, NOX-1, IL-6, and ABCA1 in macrophages, or the expression of CPT-1, MCAD-1, and other fatty acid oxidation genes in metabolic cells.

32. The method of claim 29, wherein the cellular response is cholesterol efflux from macrophages in an IRAK-1 dependent fashion.

33. The method of claim 29, wherein the cellular response is fatty acid oxidation in metabolic cells.

34. The method of claim 33, wherein the metabolic cells are hepatocytes, muscle cells, and mesangial cells.

35. The method of claim 29, wherein the cellular response is T helper cell differentiation into either T regulatory cells or T helper 17 cells in an IRAK-1 dependent fashion.
